(19) United States
(12) Patent Application Publication
(10) Pub. No.: US 2025/0107662 A1
(43) Pub. Date: Apr. 3, 2025

(54) DETERMINATION DEVICE, LEARNING DEVICE, DETERMINATION SYSTEM, DETERMINATION METHOD, LEARNING METHOD, AND PROGRAM

(71) Applicant: J-OIL MILLS, Inc., Tokyo (JP)

(72) Inventors: Yuki OKAMOTO, Tokyo (JP); Ko KIMURA, Tokyo (JP); Masami INOUE, Tokyo (JP)

(21) Appl. No.: 18/281,734

(22) PCT Filed: Mar. 11, 2022
(86) PCT No.: PCT/JP2022/010962

§ 371 Date: Sep. 12, 2023

(30) Foreign Application Priority Data
Mar. 25, 2021 (JP) 2021-051773

(51) Int. Cl. A47J 37/12; G01N 33/03

(52) U.S. Cl. CPC A47J 37/12; G01N 33/03

(57) ABSTRACT

Determination of a level of deterioration of edible oil can be realized with accuracy. A determination device for determining a level of deterioration of edible oil used in deep-fry cooking for cooking a fried food, comprising: an acquisition section configured to acquire components generating from the edible oil; a first identification section configured to identify, among the components, a content of aldehydes, Maillard reaction products, or fatty acids; and a second identification section configured to identify the level of deterioration based on the content.

### TECHNICAL FIELD

The present invention relates to a determination device, a learning device, a determination system, a determination method, a learning method, and a program.

### BACKGROUND ART

Appropriately managing the quality of edible oil in cooking of deep-fried foods (hereinafter, referred to as “cooking” or “deep-fry cooking”) preferably enables the quality of deep-fried foods to be kept. For this purpose, the conventional technique for managing the level of deterioration of edible oil has been known.

Specifically, in this conventional technique, firstly, a sensor unit detects the smell of the edible oil. When the output from the sensor for detecting the smell exceeds a predetermined threshold value, the edible oil is determined to have deteriorated, in other words, the level of deterioration increases. Thus, in the known technique for managing the level of deterioration of edible oil, the level of deterioration of edible oil can be determined without providing an oil tank with a device (for example, see Patent Literature 1).

### CITATION LIST

### Patent Literature

Patent Literature 1: JP-A-2017-136365

### SUMMARY OF INVENTION

### Technical Problem

The conventional technique uses the smell in which various components are mixed to determine the level of deterioration of edible oil. This causes a problem that the accuracy of determination of the level of deterioration of edible oil decreases when using a component having a weak correlation with the level of deterioration for the determination.

An object of the present invention is to determine the level of deterioration of edible oil with accuracy.

### Solution to Problem

In order to achieve the object above, provided is a determination device for determining a level of deterioration of edible oil used in deep-fry cooking for cooking a fried food, comprising: an acquisition section configured to acquire components generating from the edible oil; a first identification section configured to identify, among the components, a content of aldehydes, Maillard reaction products, or fatty acids; and a second identification section configured to identify the level of deterioration based on the content.

### Advantageous Effects of Invention

According to the present invention, it is possible to determine the level of deterioration with accuracy.

### BRIEF DESCRIPTION OF DRAWINGS

FIG. 1 illustrates an example of arrangement in a cooking area 1.

FIG. 2 illustrates an example of a hardware configuration of an information processing device.

FIG. 3 illustrates an example of entire processing.

FIG. 4 illustrates an example of entire processing of a configuration using AI.

FIG. 5 illustrates an example of entire processing of a configuration using a table.

FIG. 6 illustrates a relation between the content of Isobutyraldehyde and an acid value.

FIG. 7 illustrates a relation between the content of Isobutyraldehyde and color tone.

FIG. 8 illustrates a relation between the content of 2-Methylbutanal and am acid value.

FIG. 9 illustrates a relation between the content of 2-Methylbutanal and color tone.

FIG. 10 illustrates a relation between the content of 3-Methylbutanal and an acid value.

FIG. 11 illustrates a relation between the content of 3-Methylbutanal and color tone.

FIG. 12 illustrates a relation between the content of Heptanal and an acid value.

FIG. 13 illustrates a relation between the content of Heptanal and color tone.

FIG. 14 illustrates a relation between the content of 2-Nonenal and an acid value.

FIG. 15 illustrates a relation between the content of 2-Nonenal and color tone.

FIG. 16 illustrates a relation between the content of 2-PentylPyridine and an acid value.

FIG. 17 illustrates a relation between the content of 2-PentylPyridine and color tone.

FIG. 18 illustrates a relation between the content of Butanoic acid and an acid value.

FIG. 19 illustrates a relation between the content of Butanoic acid and color tone.

FIG. 20 illustrates a relation between the content of Pentanoic acid and an acid value.

FIG. 21 illustrates a relation between the content of Pentanoic acid and color tone.

FIG. 22 illustrates an example for comparison in which a relation between the content of Hexanal and an acid value is shown.

FIG. 23 illustrates an example for comparison in which a relation between the content of Hexanal and color tone is shown.

FIG. 24 illustrates an example for comparison in which a relation between the content of Octanal and an acid value is shown.

FIG. 25 illustrates an example for comparison in which a relation between the content of Octanal and color tone is shown.

FIG. 26 illustrates an example for comparison in which a relation between the content of 2,4-Decadienal and an acid value is shown.

FIG. 27 illustrates an example for comparison in which a relation between the content of 2,4-Decadienal and color tone is shown.

FIG. 28 illustrates an example for comparison in which a relation between the content of 2,4-Heptadienal and an acid value is shown.

FIG. 29 illustrates an example for comparison in which a relation between the content of 2,4-Heptadienal and color tone is shown.

FIG. 30 illustrates an example for comparison in which a relation between the content of 2 Methylpyrazine and an acid value is shown.

FIG. 31 illustrates an example for comparison in which a relation between the content of 2 Methylpyrazine and color tone is shown.

FIG. 32 illustrates an example for comparison in which a relation between the content of Pyridine and an acid value is shown.

FIG. 33 illustrates an example for comparison in which a relation between the content of Pyridine and color tone is shown.

FIG. 34 illustrates an example for comparison in which a relation between the content of Hexanoic acid and an acid value is shown.

FIG. 35 illustrates an example for comparison in which a relation between the content of Hexanoic acid and color tone is shown.

FIG. 36 illustrates an example of an information system 200.

FIG. 37 illustrates an example of a network structure.

FIG. 38 illustrates an example of a function configuration.

FIG. 39 illustrates an example of a basic configuration.

### DESCRIPTION OF EMBODIMENTS

Hereinafter, an object to be cooked using edible oil is referred to as “fried food”. The fried foods are, for example, fried chickens, croquettes, French fries, tempuras, pork cutlets, or the like.

### First Embodiment

### (Example of Arrangement in Cooking Area 1)

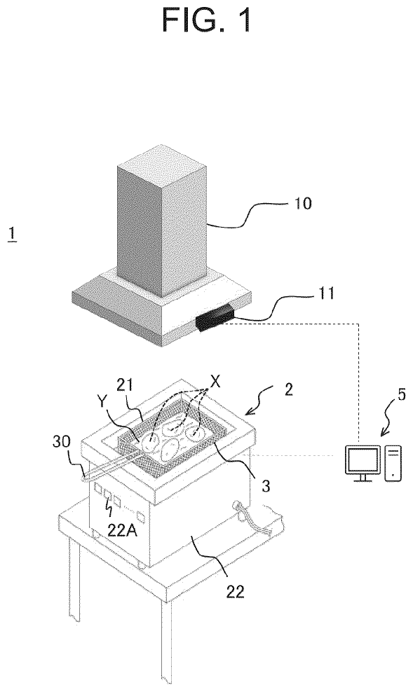

Firstly, an example of arrangement in a cooking area 1 where deep-fry cooking is performed to obtain the fried foods as listed above will be described with reference to FIG. 1.

FIG. 1 illustrates an example of arrangement in the cooking area 1. In the following, an example of edible oil is referred to as “frying oil”.

The cooking area 1 is built within a store such as a convenience store or a supermarket. The cooking area 1 is provided with cooking facilities for deep-fry cooking of deep-fried foods X. The facilities include, for example, an electric fryer 2 (hereinafter, simply “fryer 2”).

The fryer 2 is a tool equipped with an oil vat 21, a housing 22, and the like.

The oil vat 21 stores frying oil Y therein. The oil vat 21 includes, for example, a handle 30, a fry basket 3, and the like.

The housing 22 accommodates the oil vat 21 therein. On a side surface of the housing 22, switches 22A serving as a setting operation unit for setting the temperature of the frying oil Y, the details of the deep-fry cooking, or the like are provided for each type of the deep-fried foods X.

For deep-frying a food, firstly, a cook puts a deep-fried food X before deep-fried into the fry basket 3. Next, the cook immerses the fry basket 3 containing the deep-fried food X before deep-fried in the frying oil Y stored in the oil vat 21, and then hooks the handle 30 on an upper end portion of the housing 22. At the same time or around the same time, the cook presses one of the switches 22A in accordance with the type of the deep-fried food X in cooking. This operation starts the deep-fry cooking.

Upon passage of the time for completing the deep-fry cooking which corresponds to the one of the switches 22A as pressed, the fryer 2 notifies the cook of the completion of deep-frying. At the same time, the fryer 2 causes the fry basket 3 to rise from the oil vat 21 so that the deep-fried food X immersed in the frying oil is pulled up therefrom.

For informing the completion of deep-fry cooking, for example, outputting a buzzer sound from a speaker, displaying the notification on a monitor, or the like may be employed. Thus, passage of the time for completing the deep-fry cooking is notified by means of light, sound, or a combination thereof.

The cook who is aware of the completion of deep-fry cooking of the deep-fried food X pulls up the fry basket 3 to take the deep-fried food X out therefrom. Note that the fry basket 3 may be automatically pulled up by a drive mechanism.

The arrangement in the cooking area 1 is not limited to the one with the tools as illustrated in FIG. 1. For example, the fryer 2 may be any type of tool capable of cooking, and may be arranged at a place other than the position illustrated in FIG. 1.

In the cooking area 1, an imaging device for capturing an image of the frying oil Y may be installed. The imaging device is, for example, a video camera. Specifically, the video camera is installed to a ceiling or the like.

The video camera captures the surface of the frying oil Y continuously to generate images thereof. The images are generated, preferably, in the form of a movie. The video camera is installed with its condition, such as angle of view and focus, being adjusted.

Furthermore, a plurality of imaging devices may be used. Still further, the imaging device may be a camera or the like equipped in a mobile device such as a tablet or a smartphone.

In the cooking area 1, an exhaust port 10 or the like is installed so as to exhaust a substance volatilized from the fryer 2, the air, and the like. Specifically, the exhaust port 10 is installed at, for example, an upper portion of the fryer 2.

A sensor 11 is installed, for example, on the exhaust port 10 or the like. That is, the sensor 11 is preferably installed at a position allowing components volatilized from the edible oil to be sufficiently detected, for example, near the exhaust port 10.

For example, the sensor 11 is a gas sensor.

However, the sensor 11 is not limited to the type mentioned above. That is, the sensor 11 may be any type of sensor and may be installed at any position, and the number of sensors to be provided is not limited as long as it is a sensor or an odor sensor capable of analyzing the types of components and the amounts of components by mass spectrometry (GC-MS) or the like. For example, the sensor 11 may be a quartz crystal resonator sensor, a metal oxide semiconductor sensor, a membrane type surface stress sensor, a micro electro mechanical system (MEMS) semiconductor gas sensor, a portable gas analysis device, a sensor gas chromatograph, or the like.

The determination device 5 is, for example, an information processing device. The determination device 5 is connected to, for example, the sensor 11 or the like, to receive data indicating a result of analysis of a component volatilized from the edible oil. The determination device 5 may be connected to a device for analyzing a result of detection by the sensor 11.

Hereinafter, an example in which the determination device 5 is directly connected to the sensor 11 so that the determination device 5 receives a result of detection by the sensor 11 and acquires a component generating from the edible oil based on analysis or the like.

For example, the determination device 5 is configured in the manner as described below.

### (Example of Hardware Configuration of Information Processing Device)

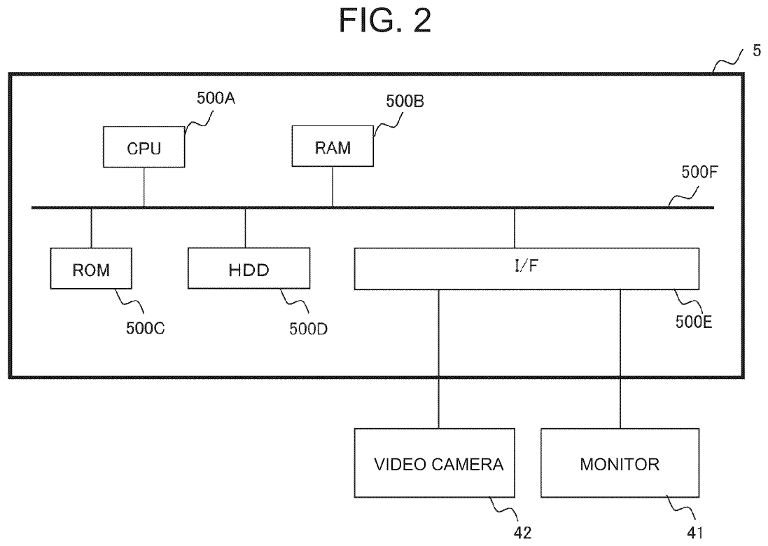

FIG. 2 illustrates an example of a hardware configuration of an information processing device. For example, the determination device 5 is an information processing device having the hardware resources as described below.

The determination device 5 includes a Central Processing Unit (hereinafter, referred to as “CPU 500A”), a Random Access Memory (hereinafter, referred to as “RAM 500B”), and the like. The determination device 5 further includes a Read Only Memory (hereinafter, referred to as “ROM 500C”), a hard disk drive (hereinafter, referred to as “HDD 500D”), interfaces (hereinafter, referred to as “I/F 500E”), and the like.

The CPU 500A is an example of a computing device and a control device.

The RAM 500B is an example of a main storage device.

The ROM 500C and the HDD 500D are examples of secondary storage devices.

The I/F 500E is provided for connection to an input device, an output device, or the like. Specifically, the I/F 500E connects an external device by wire or wireless communication for inputting and outputting data.

Note that the hardware configuration of the determination device 5 is not limited to the one described above. For example, the determination device 5 may further include a computing device, a control device, a storage device, an input device, an output device, or a secondary device. Specifically, the information processing device may include a secondary device such as an internal or external Graphics Processing Unit (GPU).

Furthermore, a plurality of learning devices 5 may be provided.

### Example of Entire Processing

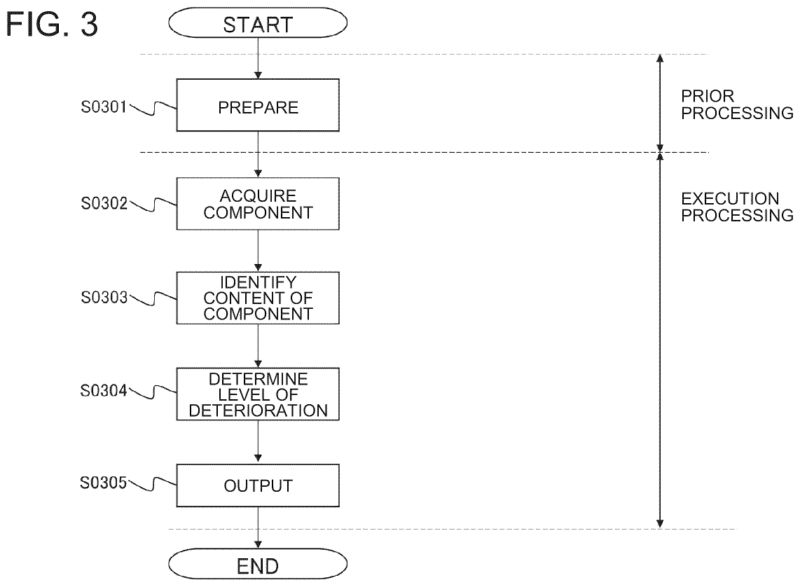

FIG. 3 illustrates an example of entire processing. For example, as illustrated in FIG. 3, the determination device executes each processing in the order of “prior processing” and “execution processing”.

The prior processing is the processing executed in advance in order to prepare for the execution processing. Specifically, in the configuration using an artificial intelligence (hereinafter, referred to as “AI”) technology, the prior processing is the processing of causing the learning model to learn. The execution processing is the processing using a learned model prepared in the prior processing.

On the other hand, the execution processing may be the processing using a table or the like. In the configuration using a table, the prior processing is the processing of preparing such as inputting the table (also referred to as a look-up table (LUT)), a formula, or the like. The execution processing is the processing using the table or the formula input in the prior processing. In the following, an example using a table will be described.

Note that the determination device does not have to execute the prior processing and the execution processing in consecutive order as illustrated in FIG. 3. In other words, the period of time for preparation by the prior processing and the period of time for executing the execution processing thereafter do not necessarily have to be consecutive.

Accordingly, in the case of using AI, after a learned model was once created, the execution processing may be performed using the learned model in other opportunities.

Furthermore, when the learned model has been already generated, the determination device may divert the learned model and omit the prior processing, and start the processing from the execution processing.

Still further, Transfer Learning, Fine tuning, or the like may be applied for a learning model and a learned model. An execution environment often varies for each device. Accordingly, while the basic configuration of AI is made learned in another information processing device, then further learning or setting may be performed by each determination device for the purpose of optimization for each execution environment.

### Example of Prior Processing

In S0301, the determination device performs preparation. The content of the prior processing differs depending on whether the configuration of the processing uses AI or a table. Note that a table, a formula, or learning data may be generated based on an operation by a user.

In the configuration using AI, the determination device performs preparation such as causing a learning model to learn or the like. On the other hand, in the configuration using a table, the determination device performs preparation such as inputting a table or the like. Details of the step of preparation will be described later.

### Example of Execution Processing

After execution of the prior processing, in other words, after completion of preparation of AI or the table, the determination device performs the execution processing, for example, in the following procedures.

In step S0302, the determination device acquires the components generating from the edible oil. Note that the determination device may acquire the components by inputting a result of analysis from another analysis device that has received a result of detection of the sensor or inputting by an operation of a user.

In step S0303, the determination device identifies the content of each component. Specifically, the determination device identifies the content of each specific component which is predetermined, such as aldehydes, Maillard reaction products, or fatty acids, among the components acquired in step S0302.

Aldehydes are, for example, Isobutyraldehyde, 2-Methylbutanal, 3-Methylbutanal, Heptanal, 2-Nonenal, etc.

Isobutylaldehyde may be also referred to as 2-methylpropanal, etc.

2-Methylbutanal may be also referred to as 2-Methylbutyraldehyde, 2-Ethylpropanal, etc.

3-Methylbutanal may be also referred to as Isovaleraldehyde, Isovaleral, Isovaleric aldehyde, 3-Methylbutyraldehyde, 3-Methylbutanal, Isopentanal, 3, 3-Dimethylpropanal, etc.

Heptanal may be also referred to as normal Heptaldehyde, 1-Heptanone, Heptyl aldehyde, Heptaldehyde, etc.

2-Nonenal may be also referred to as α-Nonenyl aldehyde, β-Hexylacrolein, 2-nonene-1-al, trans-2-Nonenal, etc.

A Maillard reaction product is 2-PentylPyridine, etc.

2-PentylPyridine may be also referred to as 2-PentylPyridine.

Fatty acids are Butanoic acid, Pentanoic acid, etc.

Butanoic acid may be also referred to as Butyric acid, n-Butyric acid, etc.

Pentanoic acid may be also referred to as valeric acid, etc.

In step S0304, the determination device determines the level of deterioration based on the content.

In step S0305, the determination device performs output based on the level of deterioration.

In the entire processing, the processing content up to step S0305 as described above differs depending on whether AI or a table is used in the configuration. Hereinafter, each configuration will be described separately.

### (Entire Processing of Configuration Using AI)

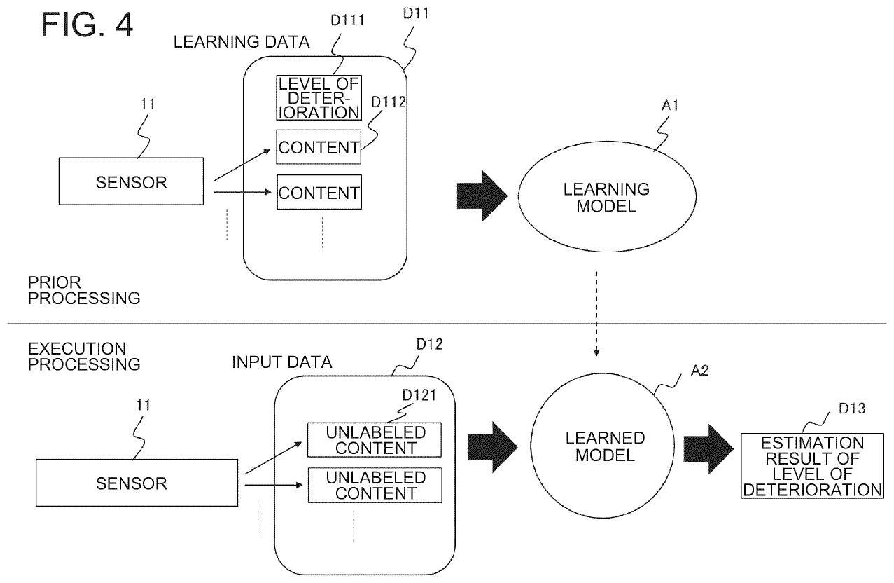

FIG. 4 illustrates an example of the entire processing of the configuration using AI. As illustrated in FIG. 4, in the configuration using AI, the prior processing is the processing of causing a learning model A1 to learn. The execution processing is the processing of determining a cooking environment or the like using a learned model A2, which is the learning model in which a certain degree of learning has been completed in the prior processing or the like.

The prior processing is, for example, the processing of causing the learning model A1 to learn using learning data D11. In other words, the prior processing is the processing of causing the learning model A1 to learn by “supervised” learning using the learning data D11 to generate the learned model A2.

The learning data DI is, for example, data obtained by combining data on, for example, content D112 and level of deterioration D111 for each component.

The level of deterioration D111 is an index indicating the increase level of deterioration of edible oil. The level of deterioration D111 will be described later.

The content D112 is the content of aldehydes, Maillard reaction products, fatty acids, or the like among the components generating from the edible oil. For example, the content D112 is expressed by an area value (system of units is dimensionless). The content D112 may be provided for each of a plurality of types.

Note that the content D112 may not be directly acquired from the sensor 11. In other words, any data format and any device can be used in inputting the content D112 as long as it is able to recognize the operations by a user or a value indicating the content and the type of the component.

As described above, upon receiving the input of the learning data D11 containing a set of the content D112 and the level of deterioration D111, the learning model A1 can learn a relation between the content D112 and the level of deterioration D111 (step S0301 in FIG. 3). Thus, the level of deterioration D111 is the information indicating the level of deterioration of the edible oil with respect to the content D112, and serves as “labeled training data” in the configuration for the “supervised” learning.

Causing the learning model A1 to learn using the learning data D11 as described above enables the determination device to learn the relation between the content D112 and the level of deterioration D111. Then, using the learned model A2 generated by this learning enables the determination device 5 to perform the following steps of the execution processing.

The execution processing is the processing of generating a result of estimation (hereinafter, simply referred to as “estimation result D13”) of the level of deterioration by AI using the input data D12 as the input.

For example, in the execution processing, the determination device inputs the input data D12 including the content (hereinafter, referred to as “unlabeled content D121”) for each component detected by the sensor 11. The execution processing differs from the prior processing in that the level of deterioration that is to be the result with respect to the content has not been known yet.

The input data D12 includes the unlabeled content D121 of one or more types. Firstly, the determination device acquires, from the sensor 11, a result of detection for the components generating from the edible oil (step S0302 in FIG. 3). Next, the determination device identifies the content of each component by analyzing the result of detection of the sensor 11 (step S0303 in FIG. 3).

When the unlabeled content D121 that is a result of identification of the content thus obtained is input to the learned model A2, the determination device can determine the level of deterioration (step S0304 in FIG. 3).

As described above, in the configuration using AI, even if the condition that differs from the condition input as the learning data D11 is input, the determination device can estimate the level of deterioration based on the learning.

### Example of Entire Processing of Configuration Using Table

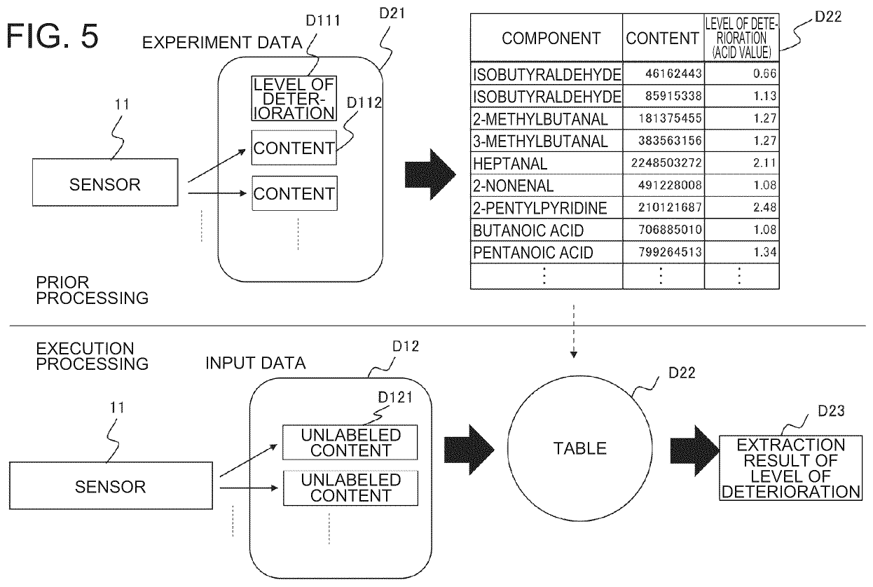

FIG. 5 illustrates an example of the entire processing of the configuration using a table. As illustrated in FIG. 5, in the configuration using a table, the prior processing is the processing of generating a table D22. The execution processing is the processing of determining the level of deterioration or the like using the table D22 generated in the prior processing.

The prior processing is, for example, the processing of gathering experiment data D21 into the format of a table.

Note that the table D22 may not be in the format of a two-dimensional table or the like. That is, in the prior processing, a format such as a formula or the like that uniquely specifies the level of deterioration D111 with respect to the content D112 may be generated.

For example, the determination device can calculate a formula representing a relation between the content D112 and the level of deterioration D111 by calculating multivariate analysis, a least-squares method, or the like on the plurality of sets of content D112 and level of deterioration D111. Hereinafter, an example in which the formula represents a straight line, in other words, a first-order expression will be described.

For example, as illustrated in FIG. 5, the table D22 is in a two-dimensional format in which the “level of deterioration” is associated with the “content” for each “component”. By referring to the table D22, the “level of deterioration” corresponding to the “content” for each “component” is determined.

The “component”, “content”, and “level of deterioration” in the table D22 are values indicated by the experiment data D21. Thus, having input the experiment data D21 in the prior processing enables the table D22 to be generated.

The experiment data D21 is the data indicating, for example, the content D112 and the level of deterioration D111 detected by the sensor 11 or the like. The content D112 and the level of deterioration D111 are, for example, the same as those in FIG. 4.

The table D22 is the data in which the content D112 is made associated with the level of deterioration D111. In the following, an example in which the level of deterioration D111 is an acid value will be described. The table D22 may include information other than the information illustrated in FIG. 5.

Using the table D22 or the formula as described above enables the determination device to make association of the content with the level of deterioration. Then, using this table D22 enables the determination device to perform the following steps in the execution processing.

For example, in the execution processing, the determination device identifies the content of each component from the sensor 11 (step S0302 and step S0303 in FIG. 3) in the same manner as in the configuration using AI.

In the same manner as the configuration using AI, in the configuration using a table as well, the execution processing differs from the prior processing in that the content of each component has not been known.

In the following, in the same manner as the configuration using AI, an example in which the input data D12 includes the unlabeled content D121 will be described.

In the same manner as the configuration using AI, when the unlabeled content D121 is input as the input data D12, the determination device extracts the corresponding level of deterioration from the table D22. In this way, the determination device outputs an extraction result D23. That is, in the same manner as the configuration using AI, the determination device extracts, from the table D22, the level of deterioration of the edible oil under the condition indicated by the input data D12 (step S0304 in FIG. 3).

As described above, in the configuration using a table, the determination device searches the level of deterioration corresponding to the condition that has been input on the table D22, thereby realizing the high-speed processing.

The determination device may estimate the level of deterioration based on linear interpolation or the like. That is, under the condition that is not input in the table D22, the determination device may calculate the level of deterioration by averaging the similar conditions in the table D22 or the like.

For example, as illustrated in FIG. 5, when “Isobutyraldehyde is 46162443” and “Isobutyraldehyde is 85915338” have been entered in the table D22 and “Isobutyraldehyde is 66038891” (that is, has not been known) is a target of the execution processing, the determination device may calculate an intermediate value between “Isobutyraldehyde is 46162443” and “Isobutyraldehyde is 85915338”.

Such interpolation enables the determination device 5 to address the condition that is not entered in the table D22.

Furthermore, the determination device may be configured to use both the table D22 and a formula. For example, the determination device extracts the level of deterioration listed in the table D22 for the condition that has been input in the table D22. On the other hand, the determination device extracts the level of deterioration calculated using the formula for the condition that has not been input in the table D22.

As can be seen from the results of experiments described below, there is a strong correlation between the content of aldehydes, Maillard reaction products, or fatty acids and the level of deterioration.

### Results of Experiments

The results of experiments conducted under the following conditions will be described as below.

### (Adjustment of Sample)

0.5 g of oil and fat were placed in a glass vial bottle for GC-MS.

### (GC-MS Analysis)

GC system: Agilent7890B (produced by Agilent Technologies, Ltd.)
Column: DB-WAX UI (produced by Agilent Technologies, Ltd., 0.25 μm, 0.25 mm×60 m)
Carrier gas: He
Ionization, measurement mode: EI, scanning
Analysis method: Analysis according to Dynamic Headspace (DHS) by Agilent Technologies, Ltd.

### (Analysis of Components of Flavor)

The area value after deconvolution of the acquired total ion chromatogram (TIC) for each component was extracted.

Deconvolution is an operation of separating overlapped peaks for each component by calculation in the software. In the analysis, a correlation between an acid value and the color tone was checked using 13 kinds of edible oil, and the components of flavor with high correlation coefficients were chosen.

### (Method of Measuring Level of Deterioration)

Method of measuring acid value: The standard methods for the analysis of fats, oils and related materials
Method of measuring color tone: Lovibond method (automatic measurement), using 1 inch cell

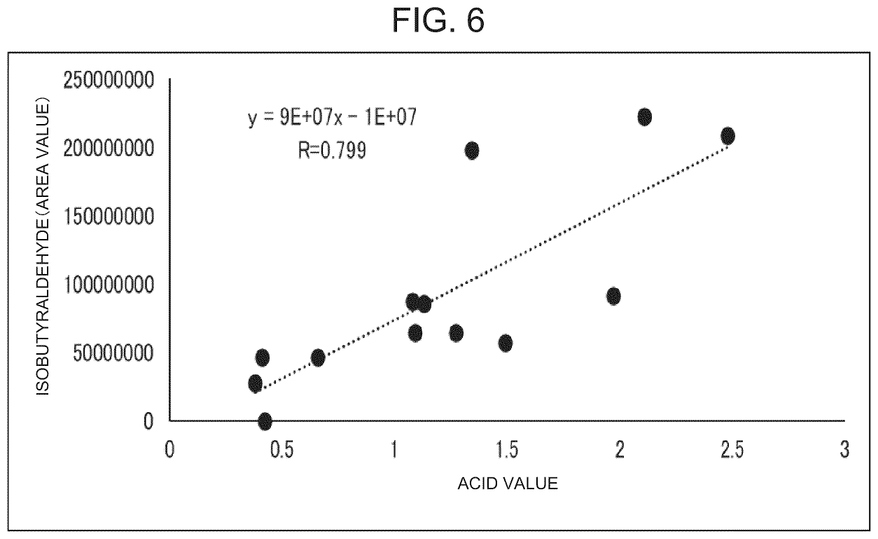

FIG. 6 illustrates a relation between the content of Isobutyraldehyde and an acid value. In FIG. 6, the vertical axis represents the content while the horizontal axis represents an acid value. FIG. 6 also illustrates a formula and straight line showing the relation between the content of Isobutyraldehyde and an acid value. The correlation is expressed using a correlation coefficient of “R”. That is, the closer “R” is to “1”, the stronger the correlation between the two variables is.

In this experiment, “R=0.799” was obtained.

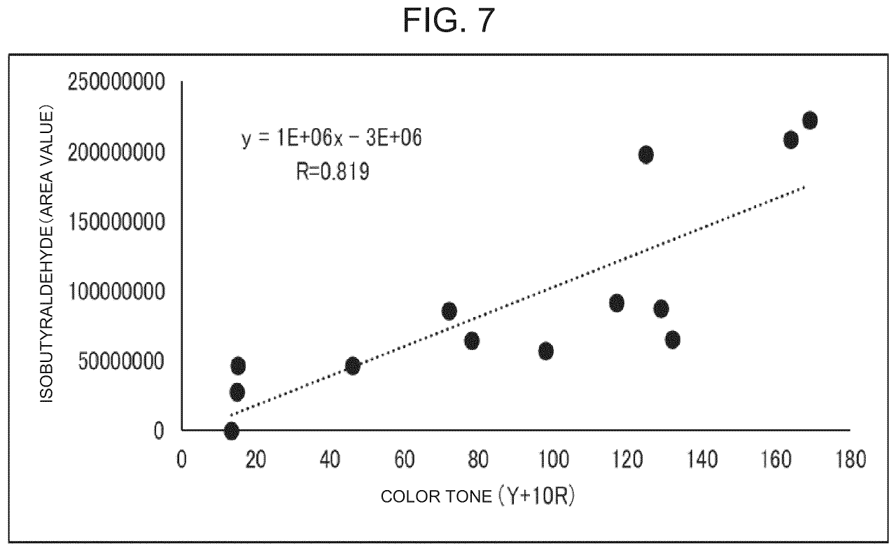

FIG. 7 illustrates a relation between the content of Isobutyraldehyde and color tone. In the same manner as FIG. 6, FIG. 7 also illustrates the results of experiments on Isobutyraldehyde. On the other hand, the experiment illustrated in FIG. 7 differs from the experiment illustrated in FIG. 6 in use of the color tone as an index of the level of deterioration. In FIG. 7, the vertical axis represents the content of component while the horizontal axis represents color tone. In the following, the points common to those in FIG. 6 will not be described.

In this experiment, “R=0.819” was obtained.

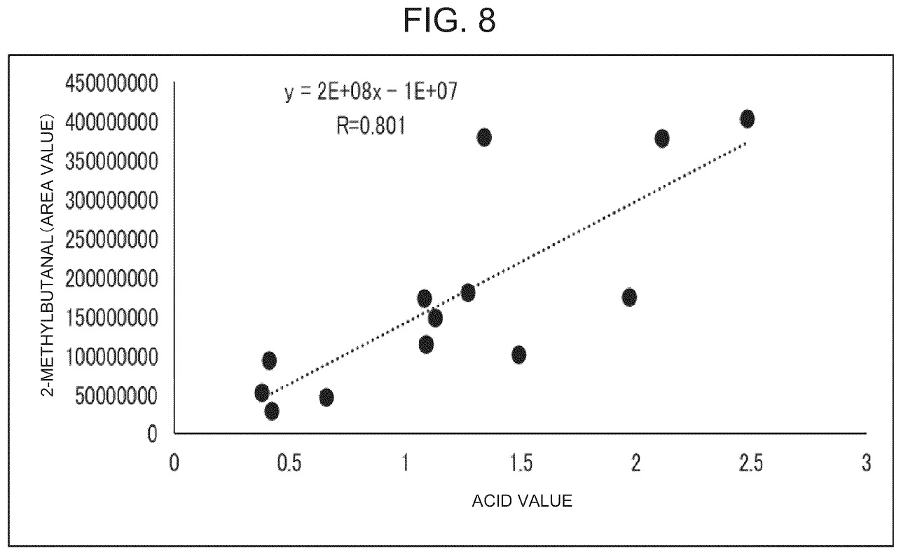

FIG. 8 illustrates a relation between the content of 2-Methylbutanal and an acid value. FIG. 8 differs from FIG. 6 in that the component is 2-Methylbutanal. In the following, the points common to those in FIG. 6 will not be described.

In this experiment, “R=0.801” was obtained.

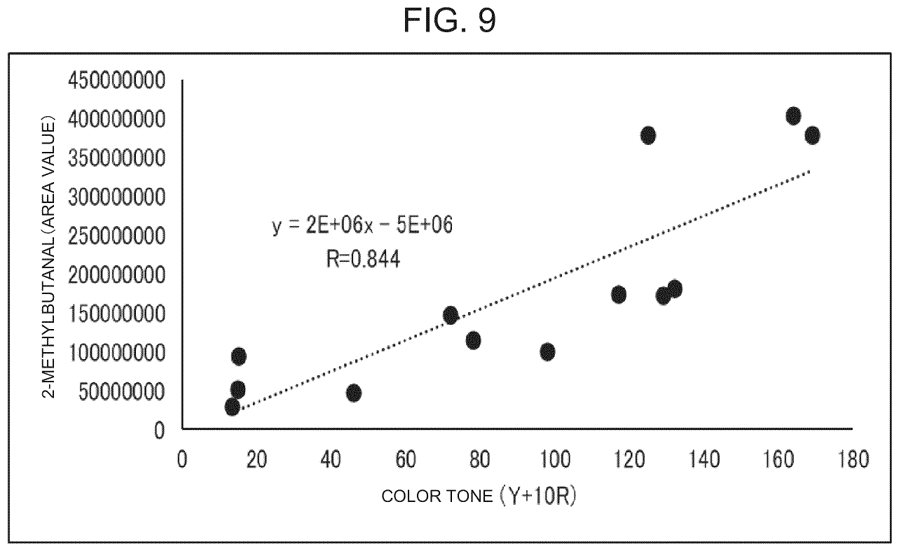

FIG. 9 illustrates a relation between the content of 2-Methylbutanal and color tone. FIG. 9 differs from FIG. 8 in use of the color tone as an index of the level of deterioration. In the following, the points common to those in FIG. 8 will not be described.

In this experiment, “R=0.844” was obtained.

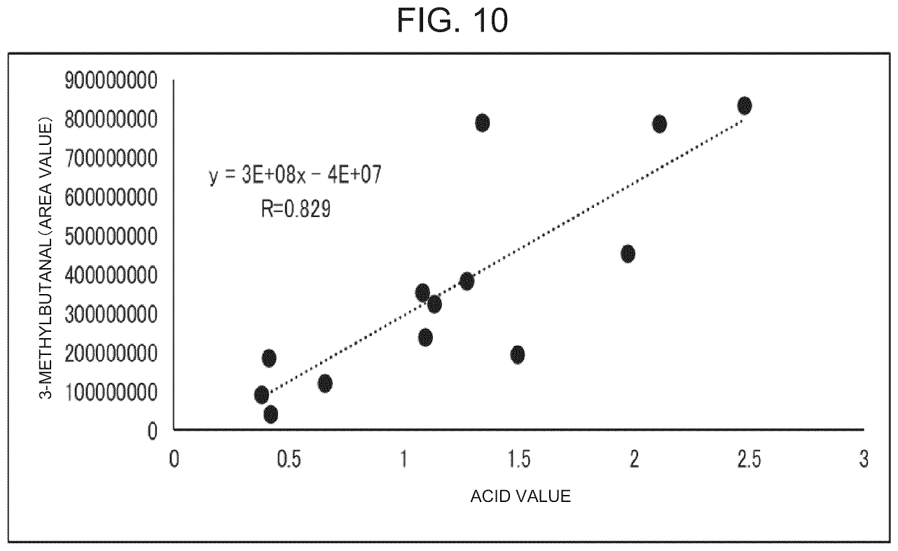

FIG. 10 illustrates a relation between the content of 3-Methylbutanal and an acid value. FIG. 10 differs from FIG. 6 in that the component is 3-Methylbutanal. In the following, the points common to those in FIG. 6 will not be described.

In this experiment, “R=0.829” was obtained.

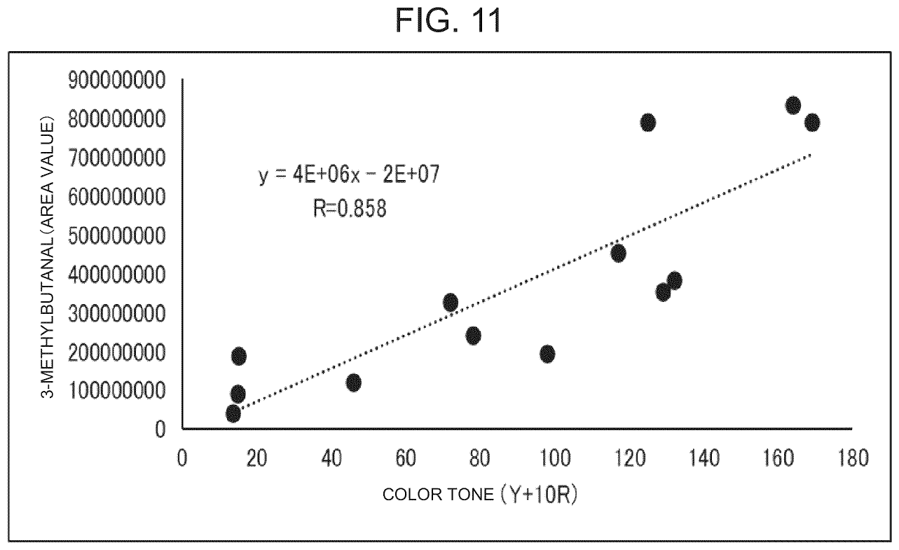

FIG. 11 illustrates a relation between the content of 3-Methylbutanal and color tone. FIG. 11 differs from FIG. 10 in use of the color tone as an index of the level of deterioration. In the following, the points common to those in FIG. 10 will not be described.

In this experiment, “R=0.858” was obtained.

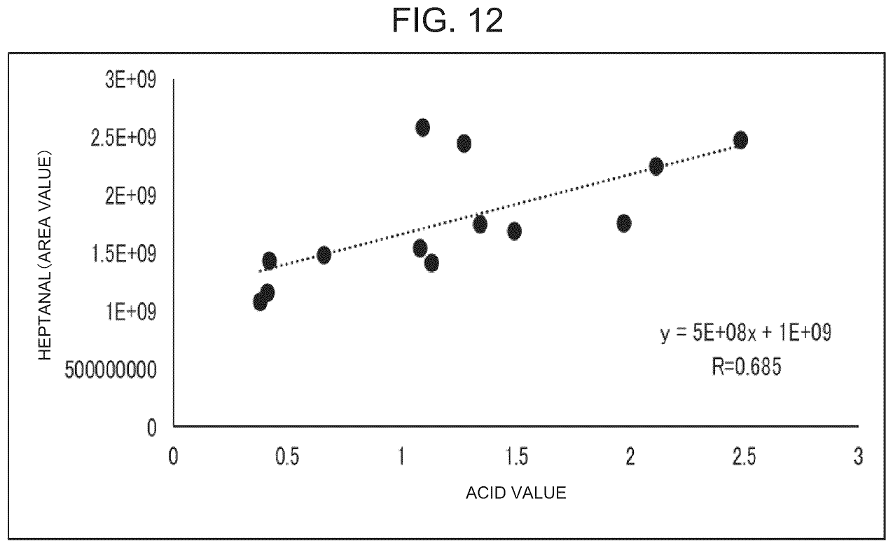

FIG. 12 illustrates a relation between the content of Heptanal and the acid value. FIG. 12 differs from FIG. 6 in that the component is Heptanal. In the following, the points common to those in FIG. 6 will not be described.

In this experiment, “R=0.685” was obtained.

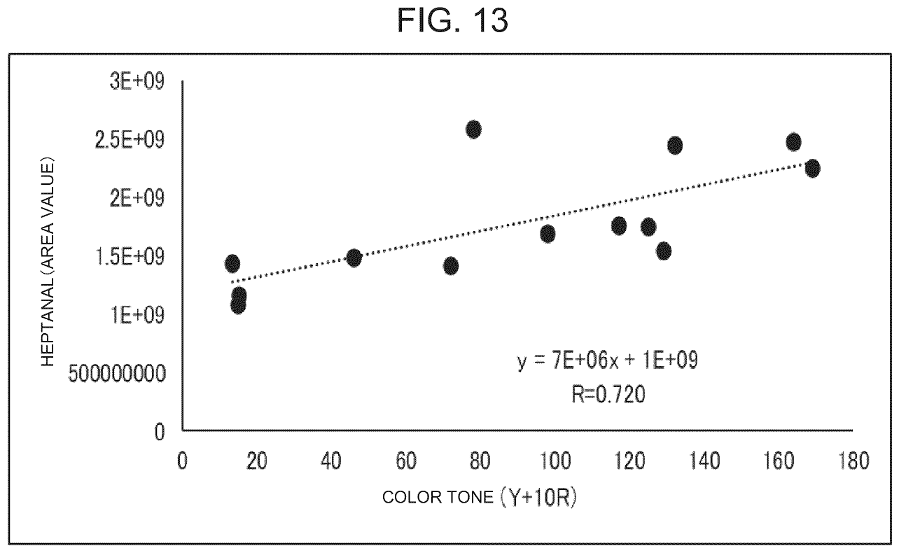

FIG. 13 illustrates a relation between the content of Heptanal and color tone. FIG. 13 differs from FIG. 12 in use of the color tone as an index of the level of deterioration. In the following, the points common to those in FIG. 12 will not be described.

In this experiment, “R=0.720” was obtained.

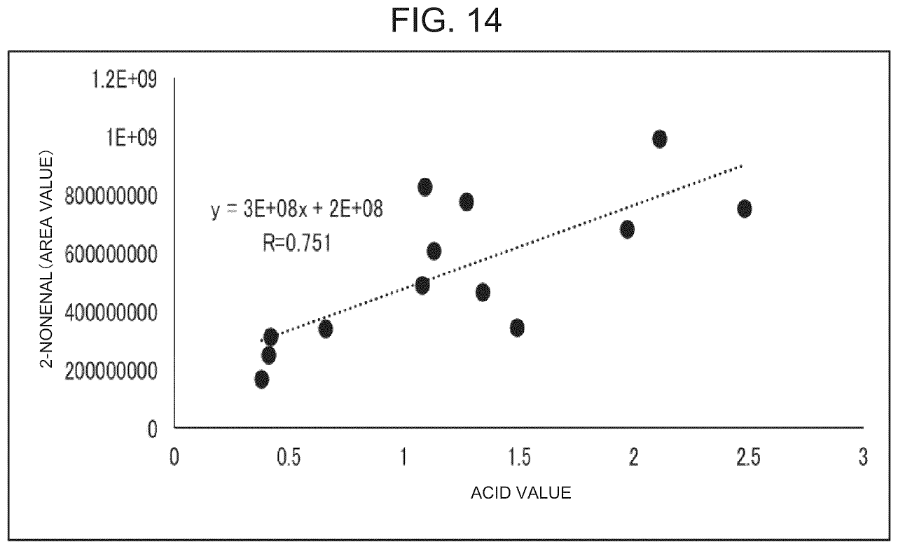

FIG. 14 illustrates a relation between the content of 2-Nonenal and an acid value. FIG. 14 differs from FIG. 6 in that the component is 2-Nonenal. In the following, the points common to those in FIG. 6 will not be described.

In this experiment, “R=0.751” was obtained.

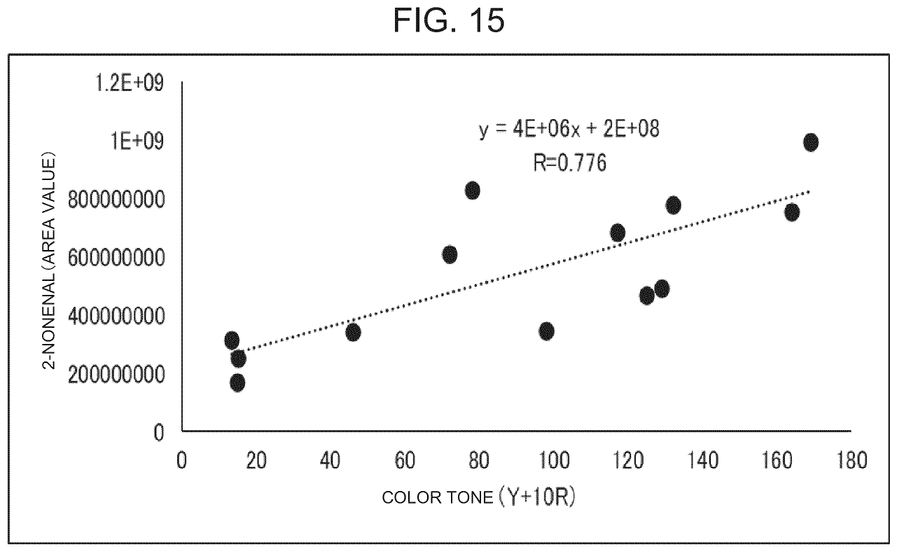

FIG. 15 illustrates a relation between the content of 2-Nonenal and color tone. FIG. 15 differs from FIG. 14 in use of the color tone as an index of the level of deterioration. In the following, the points common to those in FIG. 14 will not be described.

In this experiment, “R=0.776” was obtained.

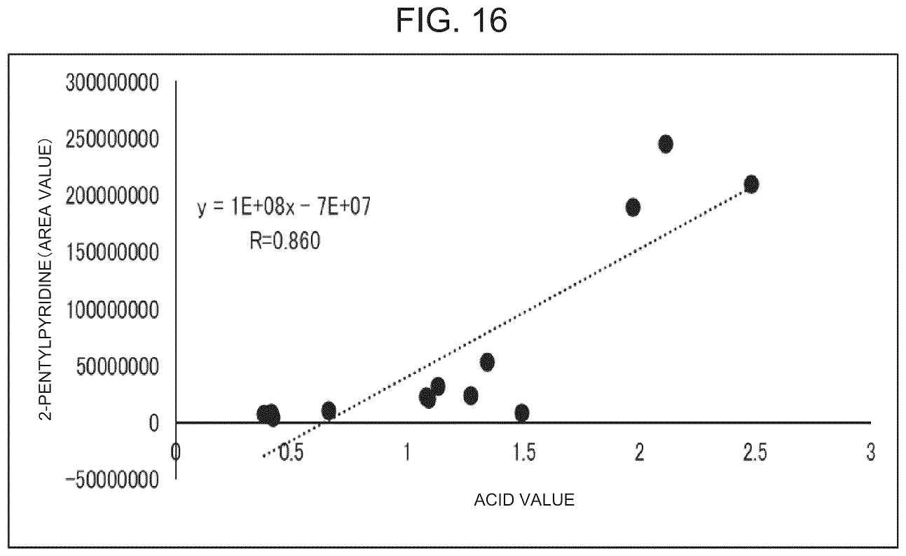

FIG. 16 illustrates a relation between the content of 2-PentylPyridine and an acid value. FIG. 16 differs from FIG. 6 in that the component is 2-PentylPyridine. In the following, the points common to those in FIG. 6 will not be described.

In this experiment, “R=0.860” was obtained.

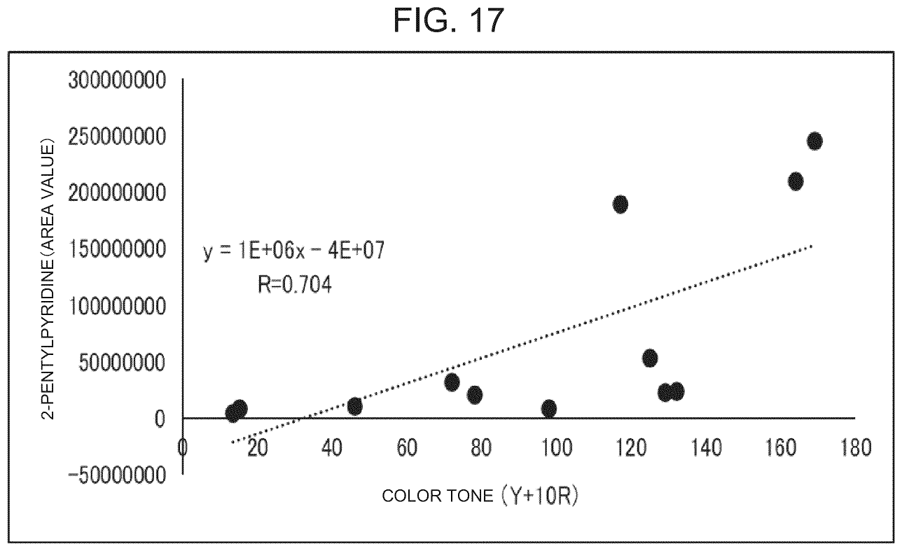

FIG. 17 illustrates a relation between the content of 2-PentylPyridine and color tone. FIG. 17 differs from FIG. 16 in use of the color tone as an index of the level of deterioration. In the following, the points common to those in FIG. 16 will not be described.

In this experiment, “R=0.704” was obtained.

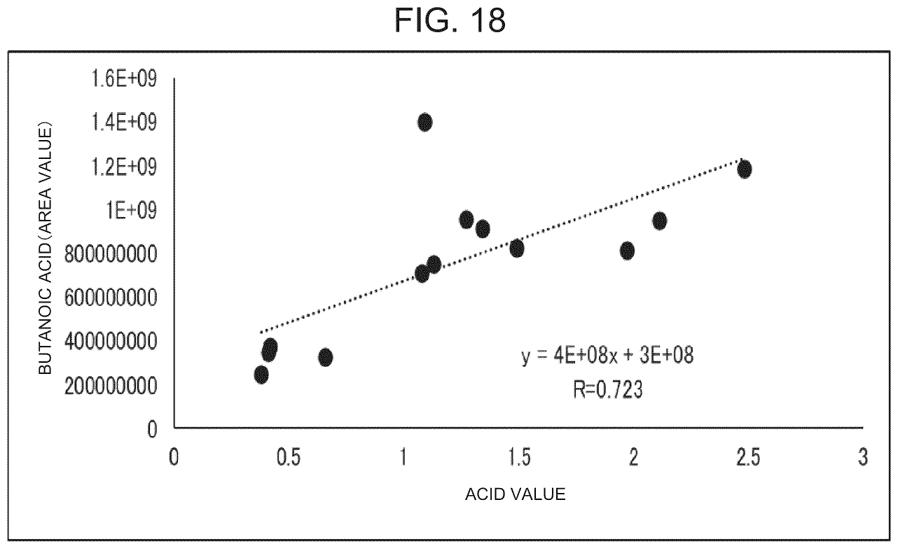

FIG. 18 illustrates a relation between the content of Butanoic acid and an acid value. FIG. 16 differs from FIG. 6 in that the component is the Butanoic acid. In the following, the points common to those in FIG. 6 will not be described.

In this experiment, “R=0.723” was obtained.

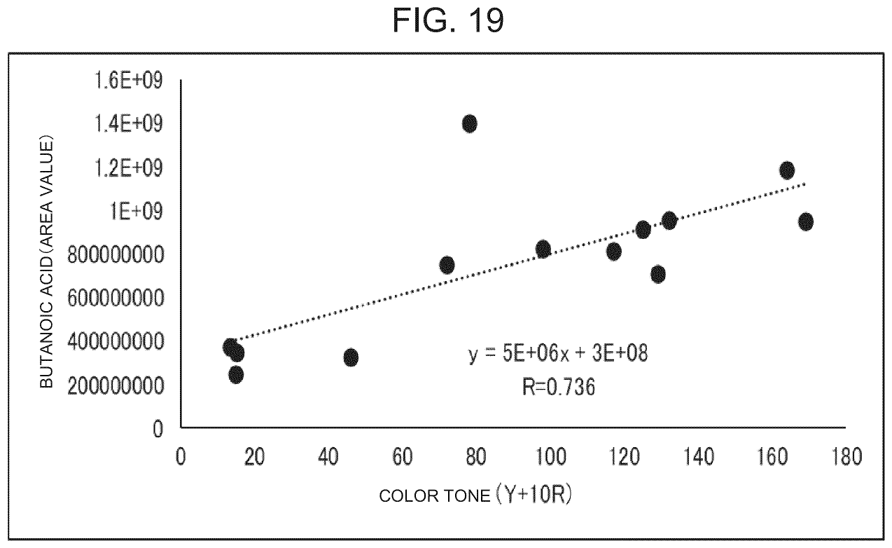

FIG. 19 illustrates a relation between the content of Butanoic acid and color tone. FIG. 19 differs from FIG. 8 in use of the color tone as an index of the level of deterioration. In the following, the points common to those in FIG. 18 will not be described.

In this experiment, “R=0.736” was obtained.

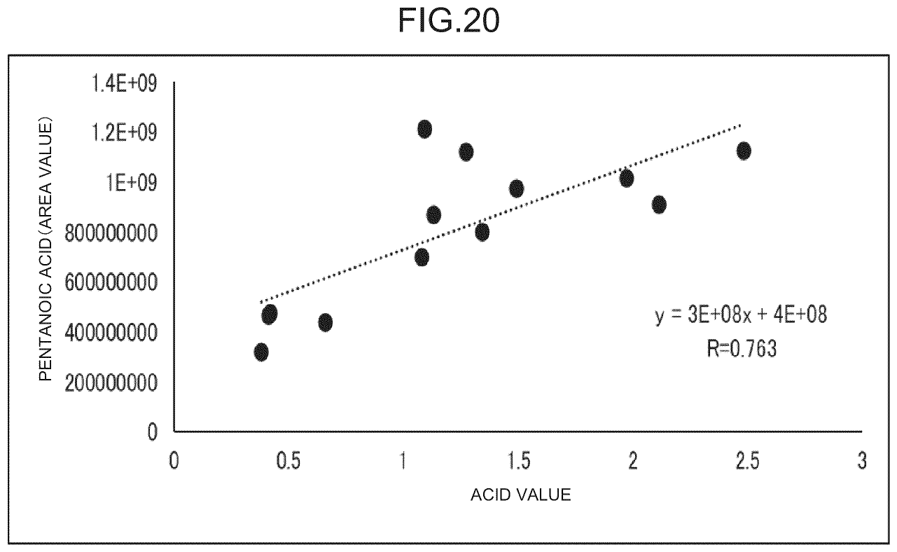

FIG. 20 illustrates a relation between the content of Pentanoic acid and an acid value. FIG. 20 differs from FIG. 6 in that the component is the Pentanoic acid. In the following, the points common to those in FIG. 6 will not be described.

In this experiment, “R=0.763” was obtained.

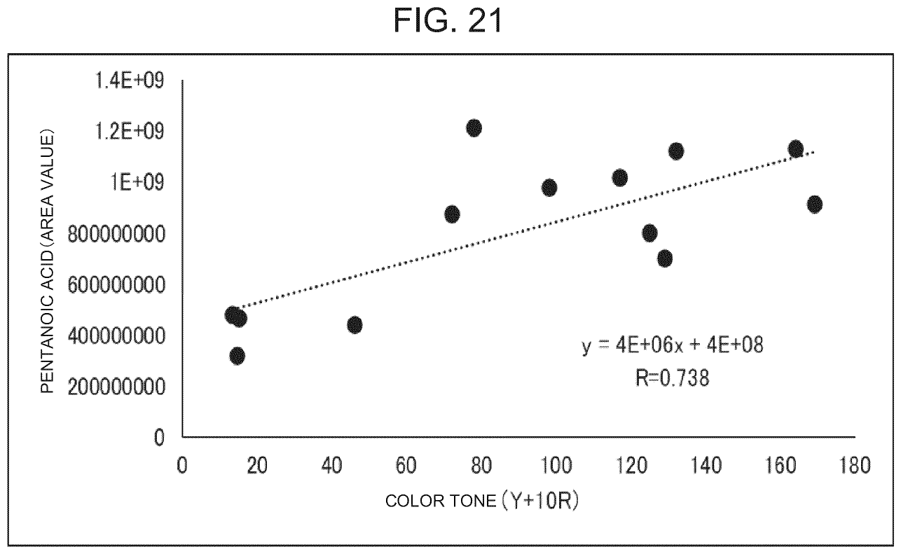

FIG. 21 illustrates a relation between the content of Pentanoic acid and color tone. FIG. 21 differs from FIG. 20 in use of the color tone as an index of the level of deterioration. In the following, the points common to those in FIG. 20 will not be described.

In this experiment, “R=0.738” was obtained.

As described in the results of experiments, the content of aldehydes, Maillard reaction products, or fatty acids is strongly correlated with the level of deterioration such as an acid value or color tone of the edible oil. This reveals that the determination device capable of identifying the content of aldehydes, Maillard reaction products, or fatty acids can accurately determine the level of deterioration of the edible oil based on calculation by AI or association using a table, or the like.

### Examples of Comparison

As described below, from edible oil, the components having weak correlations generate as well. In the following, in the same manner as the results of experiments described above, the examples in which an acid value and color tone are used for the level of deterioration will be described for each component.

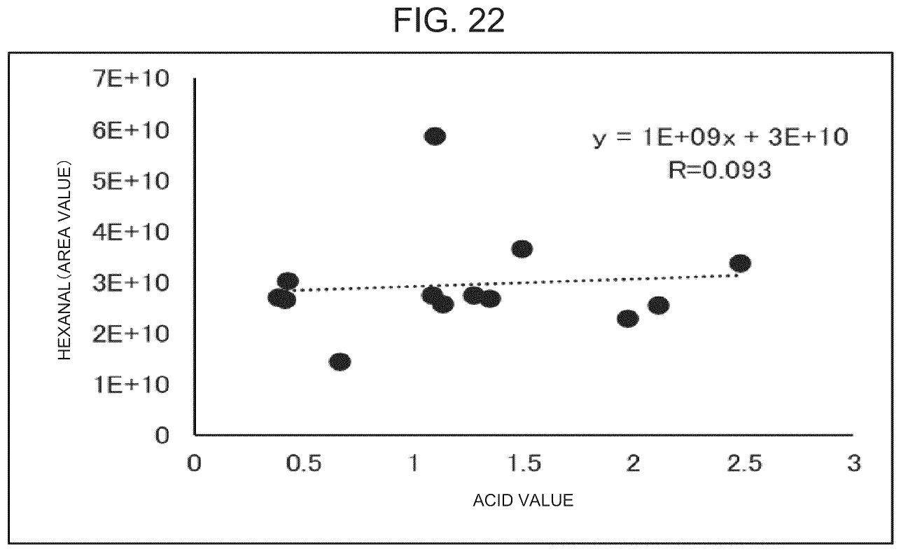

FIG. 22 illustrates an example for comparison in which a relation between the content of Hexanal and an acid value is shown.

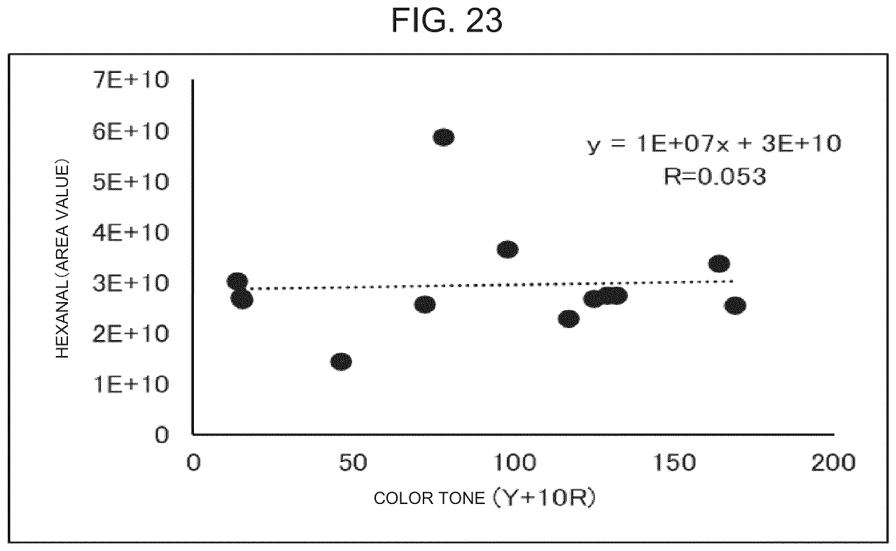

FIG. 23 illustrates an example for comparison in which a relation between the content of Hexanal and color tone is shown.

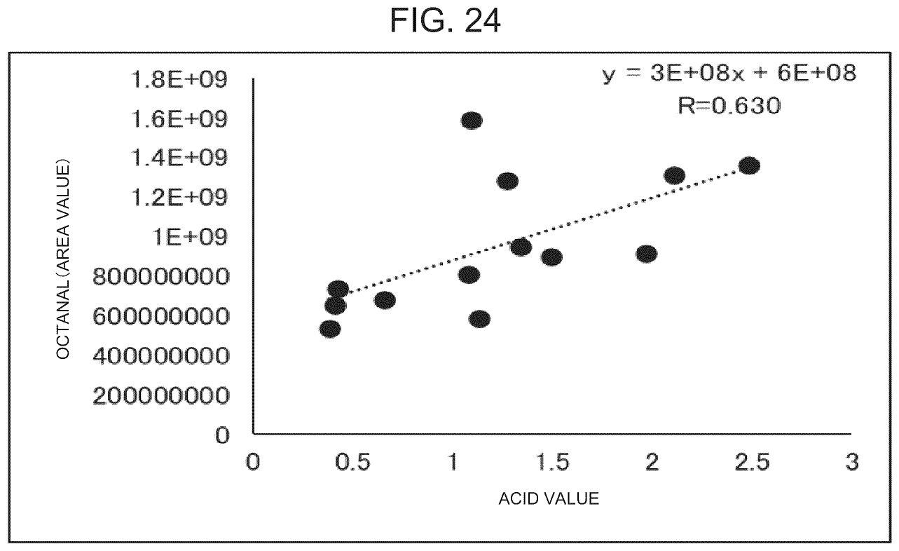

FIG. 24 illustrates an example for comparison in which a relation between the content of Octanal and an acid value is shown.

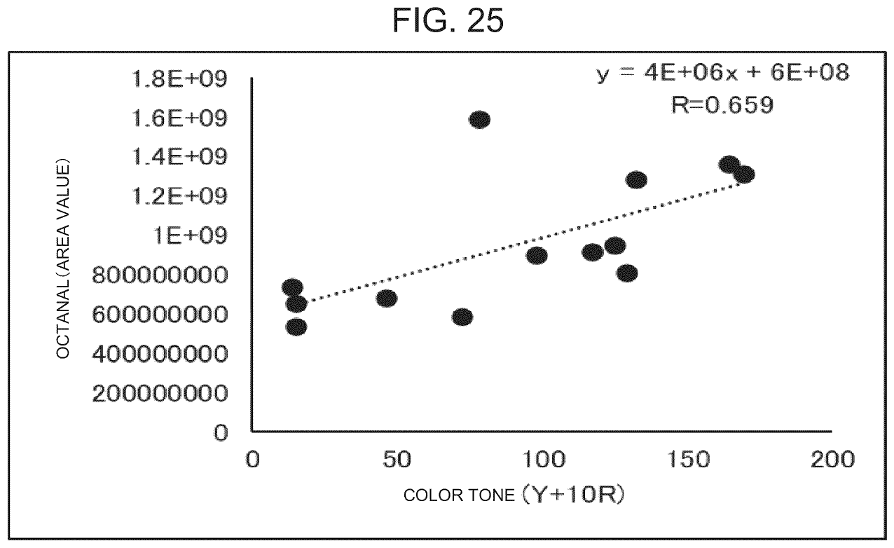

FIG. 25 illustrates an example for comparison in which a relation between the content of Octanal and color tone is shown.

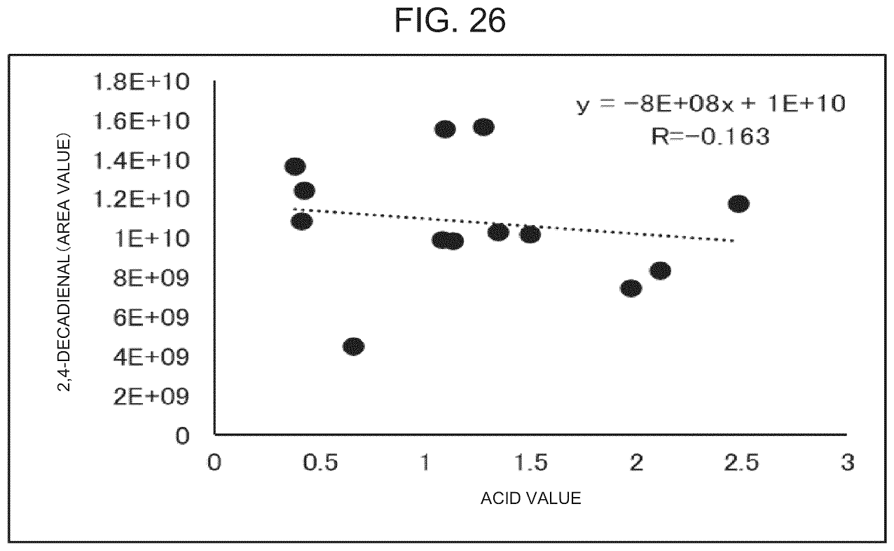

FIG. 26 illustrates an example for comparison in which a relation between the content of 2,4-Decadienal and an acid value is shown.

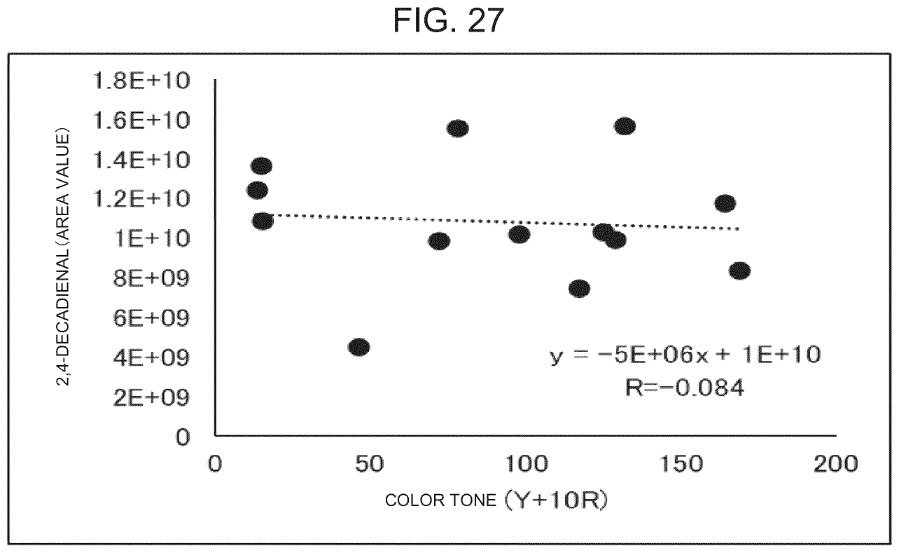

FIG. 27 illustrates an example for comparison in which a relation between the content of 2,4-Decadienal and color tone is shown.

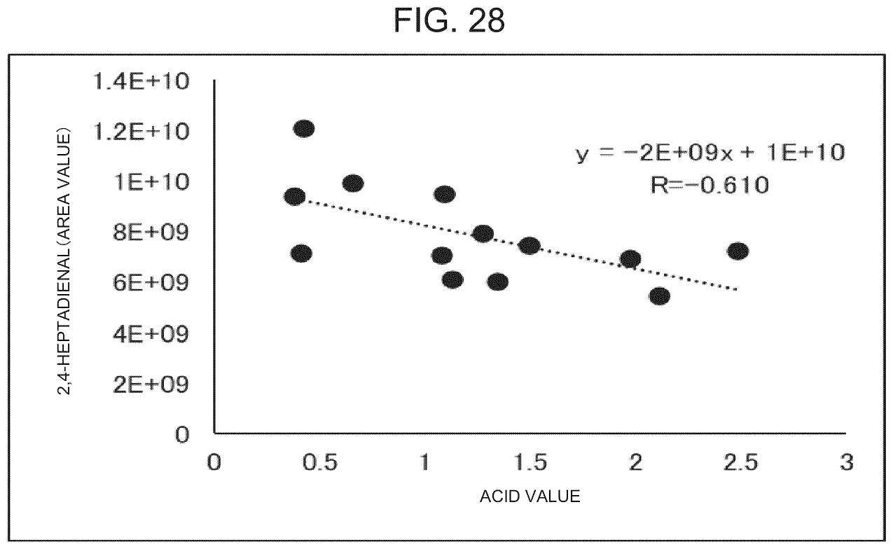

FIG. 28 illustrates an example for comparison in which a relation between the content of 2,4-Heptadienal and an acid value is shown.

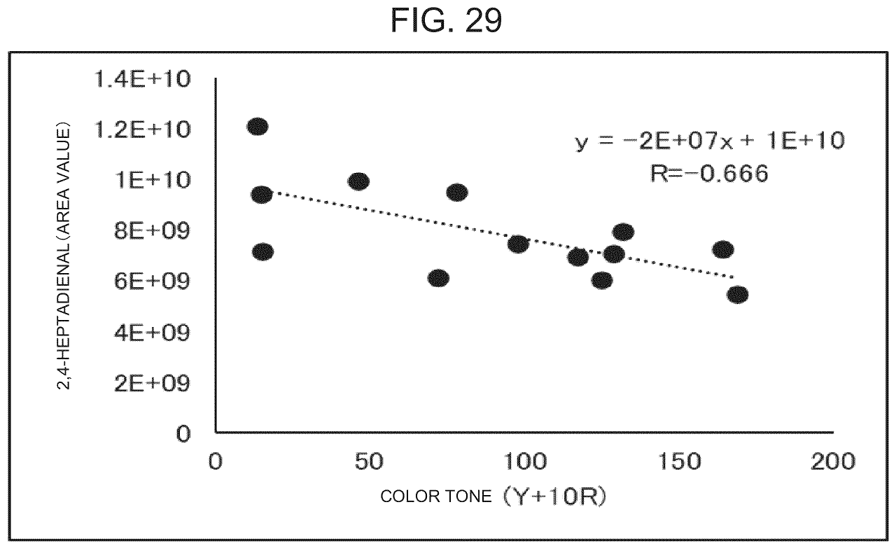

FIG. 29 illustrates an example for comparison in which a relation between the content of 2,4-Heptadienal and color tone is shown.

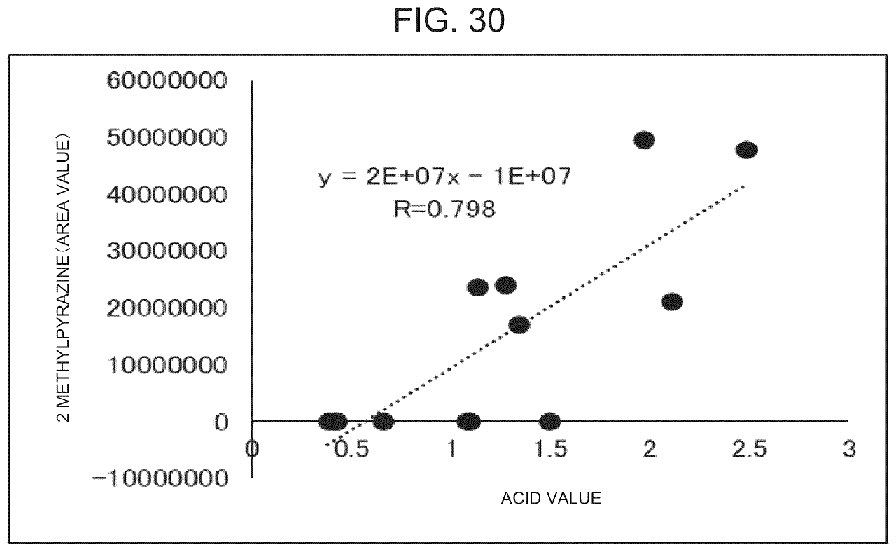

FIG. 30 illustrates an example for comparison in which a relation between the content of 2 Methylpyrazine and an acid value is shown.

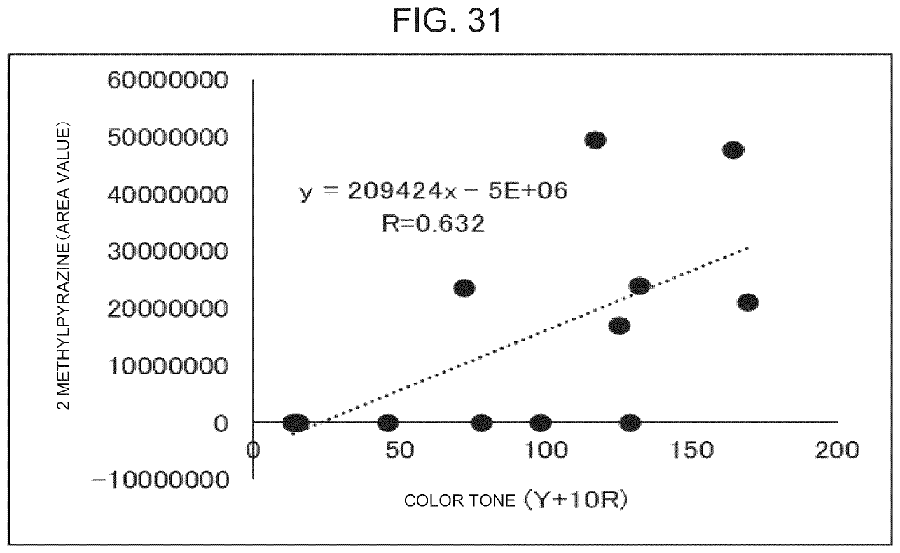

FIG. 31 illustrates an example for comparison in which a relation between the content of 2 Methylpyrazine and color tone is shown.

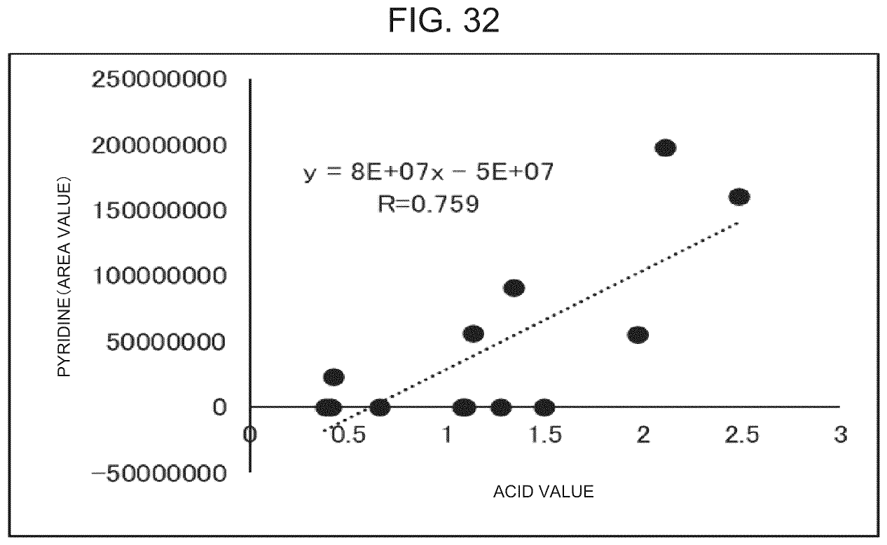

FIG. 32 illustrates an example for comparison in which a relation between the content of Pyridine and an acid value is shown.

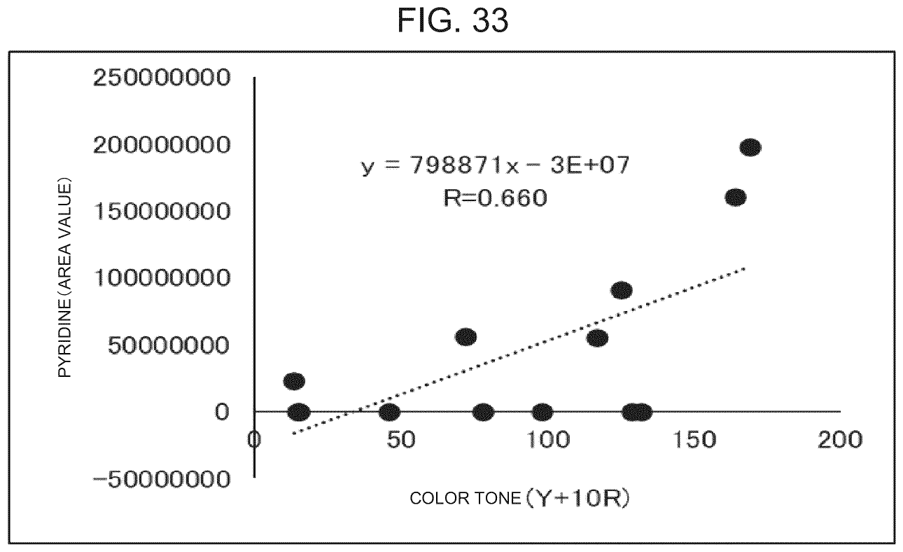

FIG. 33 illustrates an example for comparison in which a relation between the content of Pyridine and color tone is shown.

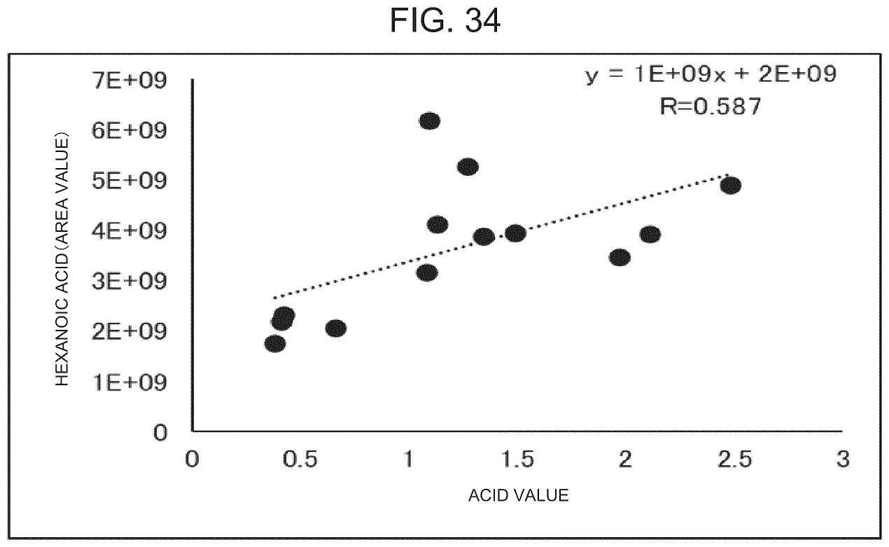

FIG. 34 illustrates an example for comparison in which a relation between the content of Hexanoic acid and an acid value is shown.

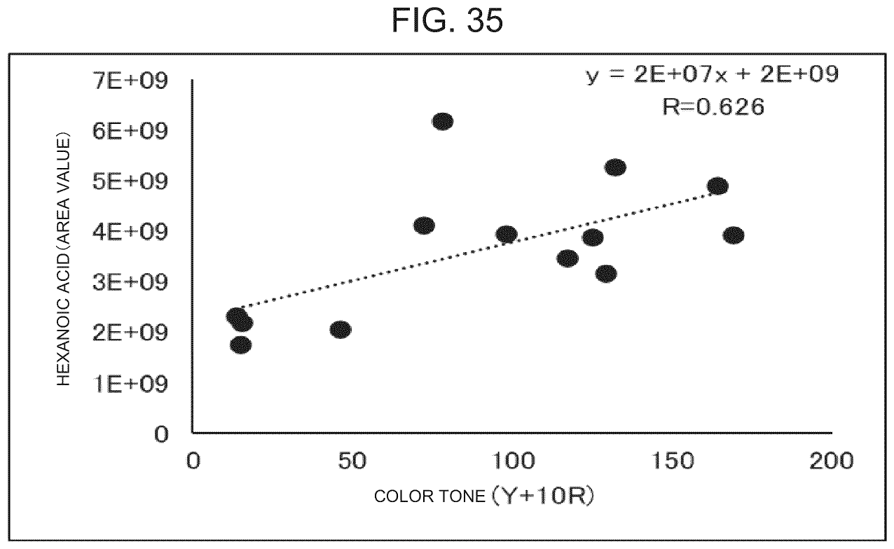

FIG. 35 illustrates an example for comparison in which a relation between the content of Hexanoic acid and color tone is shown.

In the cases of the components described in the examples for comparison, in many cases, the weak correlations are found. Knowing the content of these components having weak correlations hardly results in accurate determination of the level of deterioration.

### Second Embodiment

The second embodiment differs from the first embodiment in that, after determination of the level of deterioration, an output process, which will be described below, is performed in step S0305 of FIG. 3 based on the result of determination.

For example, upon determining that the level of deterioration of the edible oil is greater than a certain level, in other words, when the edible oil is not suitable for use in cooking, the determination device may control an adjustment unit or the like to dispose, add, or change the edible oil.

Specifically, the adjustment unit is a pump or the like. The determination device controls the adjustment unit by outputting a signal for causing the pump to operate. Thus, adjustment such as disposing, adding, or changing the edible oil can be performed in accordance with the operation of the pump.

Note that the value of the level of deterioration, which is a criterion for determining whether to perform the adjustment, is set in advance.

The determination device can maintain the edible oil with less deterioration by outputting an instruction for adjusting the edible oil based on the level of deterioration. This enables the user to provide a delicious fried food cooked with edible oil of which the level of deterioration is less.

In addition, as described below, the determination device may provide information about adjustment or the like via an information system.

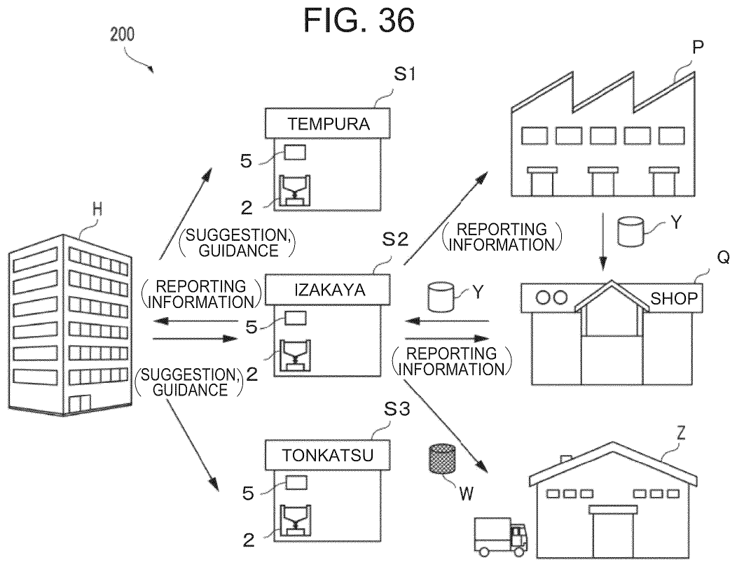

FIG. 36 illustrates an example of an information system 200. For example, the determination devices 5 installed in shops S1 to S3, respectively, are connected using a communication line or the like so as to configured the information system 200.

For example, the shop S2 (in this example, izakaya) notifies a headquarters H with reporting information. In this case, the headquarters H analyzes the number of times or frequency of receiving the reporting information. The headquarters H analyses in the same manner for the shop S1 (in this example, tempura restaurant) or the shop S3 (in this example, tonkatsu restaurant).

Based on the result of analysis thus obtained, the headquarters H provides suggestions or guidance as to whether the edible oil is appropriately used, appropriately changed, and efficiently used.

The headquarters H may manage the factories in which the fryers 2 are installed. The headquarters H may also manage each fryer 2 installed in equipment of the stores, shops, or factories.

A manufacturer P of edible oil and a seller Q of edible oil are also notified of the reporting information. Upon receiving the reporting information, the manufacturer P forms a production plan or a sales plan for edible oil. Upon receiving the reporting information, the seller Q orders and purchases edible oil from the manufacturer P. Then, the seller Q distributes the edible oil to the shop S1, shop S2, and shop S3.

Still further, a disposal company Z (note that the disposal company Z and the manufacturer P may be the same) of edible oil is also notified of the reporting information. Upon receiving the reporting information, the disposal company Z arranges collection of waste oil W. Specifically, when receiving the reporting information for a predetermined number of times, an operator from the disposal company Z visits the shop S2 to collect the waste oil W from the oil vat 21 of the fryer 2.

Still further, a cleaning company (not illustrated) may also be notified of the reporting information. Upon receiving the reporting information, a cleaning operator visits the shop S2 to clean the inside of the oil vat 21 of the fryer 2 and therearound.

Thus, using the reporting information enables quick operations including supply of edible oil, disposal thereof, and cleaning in the shops S1 to S3. Furthermore, automating the change of edible oil in the shops and stores enables reduction in the burden on a user (employee in the shops and stores). Specifically, outputting the reporting information indicating that the rate of deterioration of the edible oil exceeds a threshold value causes the edible oil in use to be changed to new one.

In the supply chain as described above, when the oil is to be adjusted, for example, by addition of oil or disposal of oil, the determination device 5 may notify the headquarters H, the disposal company Z, the manufacturer P of the amount of edible oil and the time when the oil is to be added or disposed. Thus, for addition of oil or disposal of oil, automating ordering, collection, delivery, and procedures by the information system 200 enables the user to reduce his or her workload.

### Example of Network Configuration

AI is implemented by, for example, the following network.

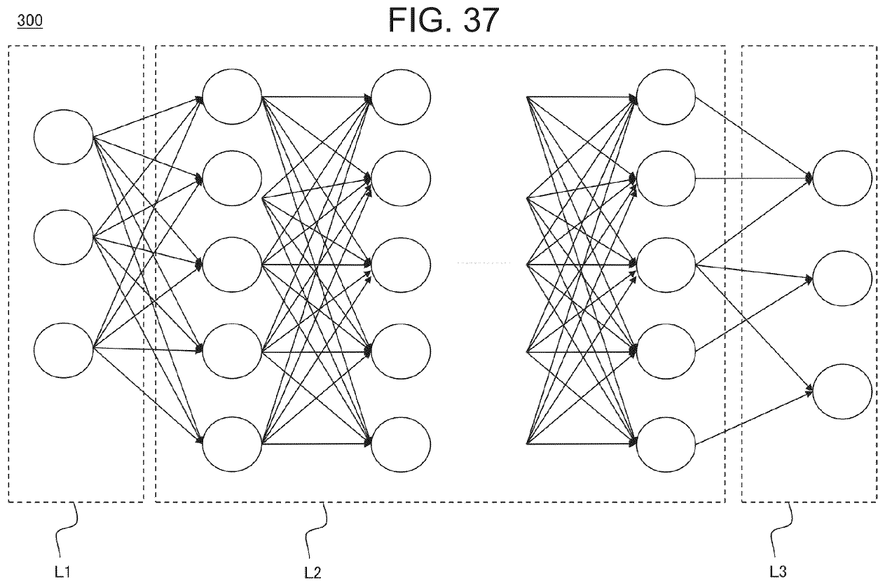

FIG. 37 illustrates an example of a network structure. For example, each of the learning model and the learned model has a network 300 having the structure as described below.

The network 300 includes, for example, an input layer L1, an intermediate layer L2 (also referred to as “hidden layer”), an output layer L3, etc.

The input layer L1 is a layer for inputting data.

The intermediate layer L2 converts the data input in the input layer L1 based on weights, biases, and the like. Thus, a result obtained by the process in the intermediate layer L2 is transmitted to the output layer L3.

The output layer L3 is a layer for outputting a result of estimation, etc.

Coefficients of the weights and the like are optimized by learning. Note that the network 300 is not limited to the network structure illustrated in FIG. 37. In other words, AI may be implemented by other machine-learning.

For example, AI may be configured to perform pre-processing such as dimensionality reduction using “unsupervised” machine-learning or the like. As shown in the results of experiments above, it is preferable that the content of a component and the level of deterioration have a correlation at a low dimension such as about the first order. In many cases, the content of a component and the level of deterioration thereof have a simple proportional relationship. Accordingly, it is preferable to calculate the level of deterioration based on a linear expression or the like using the content as an input. Using this calculation enables determination of the level of deterioration with low calculation cost and with high accuracy.

On the other hand, the 2 Pentylpyridine and the color tone often have a correlation of a quadratic expression. Accordingly, when using the correlation of 2 Pentylpyridine and the color tone, expressing the correlation by a quadratic expression allows the level of deterioration to be determined with high accuracy.

### (Deterioration)

Preferably, the level of deterioration is, for example, an acid value of edible oil, color tone of the edible oil, or a combination thereof. Compared with other indices indicating the level of deterioration, in particular, an acid value of edible oil, color tone of edible oil, or a combination thereof are strongly correlated with the content of aldehydes, Maillard reaction products, or fatty acids. According, using an acid value of edible oil, color tone of edible oil, or a combination thereof as an index of the level of deterioration enables accurate determination of the level of deterioration based on the content.

An acid value (may be referred to as “AV”) of edible oil is a value measured by a method according to, for example, the standard methods for the analysis of fats, oils and related materials, 2.3.1-2013.

The color of edible oil (may be referred to as “color tone” or “hue”) is a value measured by method according to, for example, the standard methods for the analysis of fats, oils and related materials, 2.2.1.1-2013. (for example, using a yellow component value and a red component value, a value is calculated by “the yellow component value plus 10×the red component value”).

### (Example of Function Configuration)

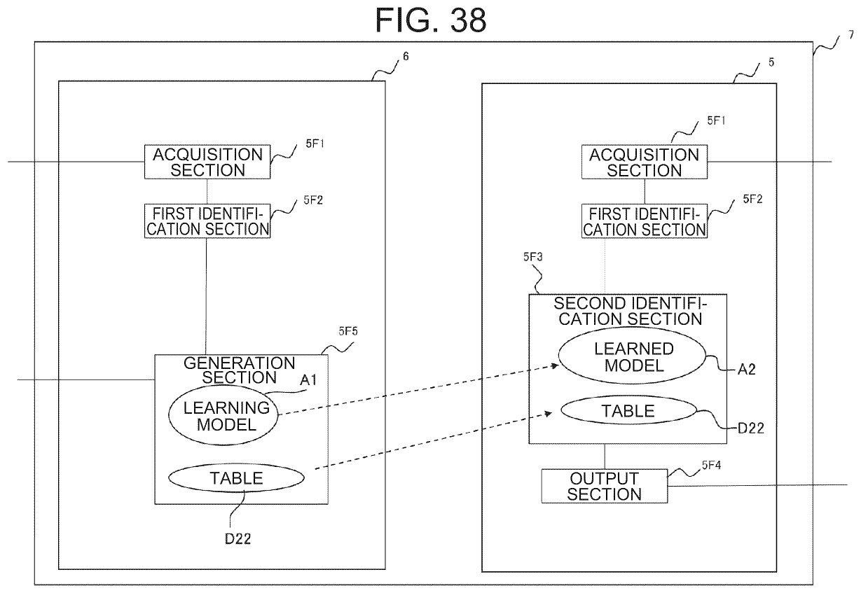

FIG. 38 illustrates an example of a function configuration. For example, the determination device 5 comprises a function configuration including an acquisition section 5F1, a first identification section 5F2, a second identification section 5F3, etc. As illustrated in FIG. 38, preferably, the determination device 5 comprises a function configuration further including an output section 5F4, etc. In the following, an example of the function configuration illustrated in FIG. 38 will be described.

The acquisition section 5F1 performs an acquisition process of acquiring the components generating from the edible oil. For example, the acquisition section 5F1 is implemented by the sensor 11, the I/F 500E, or the like.

The first identification section 5F2 performs a first identification process of identifying the content of aldehydes, Maillard reaction products, or fatty acids among the components. For example, the first identification section 5F2 is implemented by the CPU 500A or the like.

The second identification section 5F3 performs a second identification process of identifying the level of deterioration based on the content. For example, the second identification section 5F3 is implemented by the CPU500A or the like.

The output section 5F4 performs an output process of performing output based on the level of deterioration. For example, the output section 5F4 is implemented by the I/F 500E or the like.

For example, a determination system 7 including the determination device 5 and the learning device 6 comprises a function configuration as described below. In the following, an example where the learning device 6 has the hardware configuration which is the same as that of the determination device 5 will be described. However, the determination device 5 and the learning device 6 may have different hardware configurations from each other.

In the same manner as the determination device 5, the learning device 6 is, for example, a function configuration including the acquisition section 5F1, the first identification section 5F2, etc. However, the learning device 6 can employ any configuration for input or data format as long as it can enter the state and the first information. Hereinafter, the same function configurations as those of the determination device 5 will be provided with the same reference signs, and explanation thereof will be omitted.

A generation section 5F5 performs a generation process of generating the learned model A2 by causing the learning model A1 to learn. Alternatively, the generation section 5F5 performs a generation process of generating the table D22. For example, the generation section 5F5 is implemented by the CPU 500A or the like.

In the determination system 7, the learned model A2 or table D22 generated by the learning device 6 is distributed from the learning device 6 to the determination device 5 or the like via a network, etc.

The learning device 6 generates the learned model A2, the table D22, and the like by performing the prior processing. With the learned model A2 or table D22, the determination device 5 can accurately determine the level of deterioration as below based on the content of aldehydes, Maillard reaction products, or fatty acids by performing the execution processing.

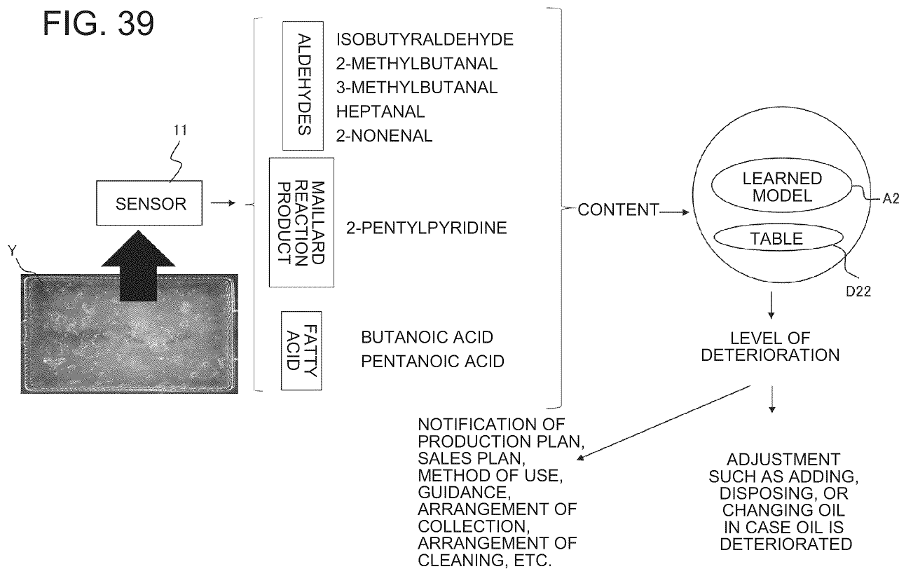

FIG. 39 illustrates an example of a basic configuration.

As illustrated in FIG. 39, various components are volatilized from the frying oil Y during cooking. These components can be detected by the sensor 11. Among these components, the content of aldehydes, Maillard reaction products, or fatty acids is focused.

In advance, AI learns a relation between the content of aldehydes, Maillard reaction products, or fatty acids and the level of deterioration to generate the learned model A2. Alternatively, a relation between the content of aldehydes, Maillard reaction products, or fatty acids and the level of deterioration is obtained using the table D22 or the like. Using the learned model A2, table D22, or the like enables the determination device to accurately determine the level of deterioration based on the content of aldehydes, Maillard reaction products, or fatty acids.

In this case, in particular, the determination device can accurately determine the level of deterioration more than the case of, for example, determining the level of deterioration based on the content of other components.

The content may be a combination of the content of a plurality of components. For example, using two or more types from aldehydes, Maillard reaction products, or fatty acids enables the determination device to determine the level of deterioration with higher accuracy. Specifically, when using the content of two or more types of components, the weight for the component of which the content is the highest is made increased. In particular, when increasing the weight for the component, such as 3-Methylbutanal, having a particularly strong correlation, enables the determination device to determine the level of deterioration with higher accuracy.

Furthermore, when using the content of two or more types of components, it is preferable to choose the components having a weak correlation among the components. For example, when using a combination of the two components, it is preferable to employ 3-Methylbutanal and Pentanoic acid, or the like.

A correlation between 3-Methylbutanal and Pentanoic acid is “0.535”. On the other hand, a correlation between 3-Methylbutanal and 2 Pentylpyridine is “0.781”. Comparison between them shows that the correlation between the combination of 3-Methylbutanal and Pentanoic acid is weaker than the other.

When determination is made based on regression analysis or the like, multicollinearity occurs depending on a combination of components to be selected. In other words, in a combination of components having strong correlation, the components often show the similar tendency, which do not help for reference. Accordingly, preferably, the determination device uses a combination of components having a weak correlation.

In the multiple regression analysis using a combination of 3-Methylbutanal and Pentanoic acid and using an acid value as an objective variable, the multiple correlation coefficient is “0.911”. On the other hand, in the multiple regression analysis using a combination of 3-Methylbutanal and 2-Pentylpyridine and using an acid value as an objective variable, the multiple correlation coefficient is “0.896”. This shows that, using a combination of components having a weak correlation, such as each of the content of 3-Methylbutanal and Pentanoic acid enables the level of deterioration to be determined more accurately based on the much stronger correlation.

### Modifications

A part or all of the parameters may be acquired through an input operation or the like by a user.

In the determination, for example, the shelf life, weight of a fried food, temperature, humidity, size, arrangement of fried foods during deep-fry cooking, thickness, ratio of batter-coating, or a combination thereof may be considered.

The result of determination may be output in the format of indicating the tendency of deterioration or in the format in which when to change the edible oil, such as whether it is time to change the edible oil, is estimated.

Specifically, the result of determination is displayed on the monito in the format like “current level of deterioration is 00%”. That is, the monitor shows the current level expressed by a percentage where the percentage of the time in the future to change the oil is “100%”. On the other hand, when the level of deterioration shows the time to change has been reached, the monitor may display, for example, the result of determination by displaying a message like “please change frying oil”.

When the type of fried food to be deep-fried next and the number of pieces for each type are predetermined, the monitor displays, for example, “∘ more pieces to be deep-dried are left”, “you can deep-fry o pieces of ∘∘ or ● pieces of ●● for next time”, “add new oil now, and you can use this oil for o more days”, and the like. That is, the monitor may display the result of determination so as to inform the details capable of being cooked until the time to change the oil has been reached, which are, for example, the type and number of fried foods.

Capturing an image of the edible oil and analyzing it may enable calculation of the “number of bubbles”, “size of a bubble”, “ratio of the area where bubbles each having the predetermined size are formed relative to the total area”, “time from formation of the specific bubbles to disappearance thereof (speed of disappearance)”, or a combination thereof.

Furthermore, using these results of calculation, images, or a combination thereof may enable identification of the “acid value”, “color tone”, “rate of increase in viscosity”, “degree of flow of bubbles”, “visibility of the outline in an image of a fried food”, type of frying oil, type of a fried food, quantity of fried foods, a combination thereof, or the like.

### Other Embodiments

The determination device may be further connected to various sensors such as a microphone, a thermometer, an optical sensor, etc. Using various sensors described above enables more accurate detection of components.

In the examples described above, the determination device performs both the prior processing on the learning model and the execution processing using the learned model. However, the prior processing and the execution processing may not be performed by the same information processing device. Furthermore, each of the prior processing and the execution processing may not be executed consistently in one information processing device. In other words, each of the processing, storing data, and the like may be performed by an information system or the like including a plurality of information processing devices.

Note that the determination device or the like may further perform additional learning after the execution processing or before the execution processing.

Other embodiments in which the embodiments described above are combined each other may be adopted.

In an exemplary embodiment, the process of reducing over-learning (also referred to as “over-fitting”) such as dropout may be performed. In addition, the pre-processes such as dimension reduction and normalization may be performed.

The network architectures of the learning model and learned model may be CNN. For example, the network architecture may have a configuration such as RNN (Recurrent Neural Network) or LSTM (Long Short-Term Memory). In other words, the network architecture of AI may be other than deep-learning.

Furthermore, the learning model and the learned model may have a configuration including hyper parameters. That is, the learning model and the learned model may allow a user to set a part of the settings. Still further, AI may identify the amount of feature to be trained, or the user may set some or all of the amount of feature to be trained.

Still further, the learning model and the learned model may use other types of machine learning. For example, the learning model and the learned model may perform the pre-processing such as normalization by using an unsupervised model. Furthermore, learning may be reinforcement learning, or the like.

In learning, data expansion or the like may be performed. In other words, in order to increase the training data to be used in learning of the learning model, a pre-processing for expanding one piece of experiment data into a plurality of pieces of learning data may be performed. Thus, increasing the training data can advance the learning of the learning model more.

The present invention may be implemented by the determination and learning method exemplified above or a program for executing the processing equivalent to the processing described above (including firmware and one equivalent to the program, and hereinafter, simply referred to as “program”).

That is, the present invention may be realized by a program or the like described in a programming language or the like so that a predetermined result is obtained by executing a command to a computer. The program may be configured to execute a part of the processing by hardware such as an integrated circuit (IC) or a computing device such as a GPU.

The program causes a computer to execute the processing described above by making the computing device, control device, and storage device equipped in the computer cooperate. That is, the program is loaded onto the main storage device or the like and then issues a command to cause the computing device to execute the calculation, thereby causing the computer to operate.

Furthermore, the program may be provided via a computer-readable recording medium or a telecommunication line such as a network.

The present invention may be realized by a system including a plurality of devices. That is, the information processing system by a plurality of computers may execute the processing described above by a redundant, parallel, or distributed system, or a combination thereof. Accordingly, the present invention may be realized by a device having a configuration other than the hardware configuration described above and a system other than the one described above.

In the above, the present invention has been described with reference to the embodiments of the present invention. The present invention is not limited to the embodiments described above, and various modifications may be made therein. For example, each of the embodiments is described in detail herein for the purpose of clarity and a concise description, and the present invention is not necessarily limited to those including all the features described above. Furthermore, some of the features according to a predetermined embodiment can be replaced with other features according to the separate embodiments, and other features can be added to the configuration of a predetermined embodiment. Still further, some of the features can include other features of the separate embodiments, be deleted, and/or replaced.

### REFERENCE SIGNS LIST

5: determination device
5F1: acquisition section
5F2: first identification section
5F3: second identification section
5F4: output section
5F5: generation section
6: learning device
7: determination system
11: sensor
300: network
A1: learning model
A2: learned model
D11: learning data
D111: level of deterioration
D112: content
D12: input data
D121: unlabeled content
D13: estimation result
D21: experiment data
D22: table
D23: extraction result
L1: input layer
L2: intermediate layer
L3: output layer

1. A determination device for determining a level of deterioration of an edible oil used in deep-fry cooking for cooking a fried food, comprising:
an acquisition section configured to acquire components generating from the edible oil;
a first identification section configured to identify, among the components, a content of aldehydes, Maillard reaction products, or fatty acids; and
a second identification section configured to identify the level of deterioration based on the content.

2. The determination device according to claim 1, wherein
the level of deterioration is an acid value or a color tone of the edible oil.

3. The determination device according to claim 1, wherein
the aldehydes include Isobutyraldehyde, 2-Methylbutanal, 3-Methylbutanal, Heptanal, or 2-Nonenal.

4. The determination device according to claim 1, wherein
the Maillard reaction products include 2-Pentylpyridine.

5. The determination device according to claim 1, wherein
the fatty acids include Butanoic acid or Pentanoic acid.

6. The determination device according to claim 1, further comprising an output section configured to perform output based on the level of deterioration.

7. The determination device according to claim 6, wherein
the output section is configured to output a notification for ordering the edible oil, a notification for making a production plan or a sales plan of the edible oil, a notification for providing suggestion or guidance of a method of use of the edible oil, a notification for arranging collection of waste oil, or a notification for arranging cleaning of an oil vat in which the edible oil is stored.

8. The determination device according to claim 6, wherein
the output section is configured to control an adjustment unit for performing adjustment of disposing, adding, or changing the edible oil so as to make the adjustment unit to perform the adjustment when the edible oil is equal to or more than a certain level of deterioration.

9. A learning device comprising:
an acquisition section configured to acquire components generating from an edible oil used in deep-fry cooking for cooking a fried food;
a first identification section configured to identify, among the components, a content of aldehydes, Maillard reaction products, or fatty acids; and
a generation section configured to enter the content and a level of deterioration of the edible oil and cause a learning model to learn, so as to generate a learned model.

10. A determination system comprising:
the determination device according to claim 1; and
the learning device according to claim 9.

11. A determination method of determining a level of deterioration of an edible oil used in deep-fry cooking for cooking a fried food, comprising:
an acquisition step of acquiring components generating from the edible oil;
a first identification step of identifying, among the components, a content of aldehydes, Maillard reaction products, or fatty acids; and
a second identification step of identifying the level of deterioration based on the content.

12. A program for making a computer execute the determination method according to claim 11.

13. A learning method comprising:
an acquisition step of acquiring components generating from an edible oil used in deep-fry cooking for cooking a fried food;
a first identification step of identifying, among the components, a content of aldehydes, Maillard reaction products, or fatty acids; and
a generating step of generating a learned model by entering the content and a level of deterioration of the edible oil and causing a learning model to learn.

14. A computer-readable media for making a computer execute the learning method according to claim 13.
